(19) United States
(12) Patent Application Publication
(10) Pub. No.: US 2026/0069521 A1
(43) Pub. Date: Mar. 12, 2026

(54) COSMETIC COMPOSITION FOR IMPROVED PENETRATION

(71) Applicant: L'OREAL, Paris (FR)

(72) Inventors: Nicole BURKHARD, Ledgewood, NJ (US); Stephanie MORRIS, Montclair, NJ (US); Mark ZAW, Inselin, NJ (US); Stephen LYNCH, Westfield, NJ (US); Jennifer PAULUCCI, Clark, NJ (US); Lauren GUINAW, Clark, NJ (US)

(73) Assignee: L'OREAL, Paris (FR)

(21) Appl. No.: 19/308,486

(22) Filed: Aug. 25, 2025

Related U.S. Application Data
(60) Provisional application No. 63/693,745, filed on Sep. 12, 2024.

(30) Foreign Application Priority Data
Nov. 8, 2024 (FR) 2412244
Dec. 29, 2024 (WO) PCT/US2024/062216

(51) Int. Cl. A61K 8/42; A61K 8/34; A61K 8/37; A61K 8/44; A61K 8/49; A61K 8/73; A61K 8/86; A61K 8/891; A61Q 19/00

(52) U.S. Cl. CPC A61K 8/42; A61K 8/342; A61K 8/37; A61K 8/44; A61K 8/4913; A61K 8/494; A61K 8/73; A61K 8/86; A61K 8/891; A61Q 19/008

(57) ABSTRACT

The instant disclosure relates to compositions, especially cosmetic composition, in particular for caring for keratin materials. The cosmetic compositions include at least one skin penetration enhancer; at least three natural moisturizing factors; hydroxyethylpiperazine ethane sulfonic acid. The compositions are useful in methods for improving the appearance of keratin materials.

### CROSS-REFERENCE TO RELATED APPLICATIONS

This application claims benefit of U.S. Ser. No. 63/693,745 filed Sep. 12, 2024, French Application No. 2412244 filed Nov. 8, 2024, and International Patent Application No. PCT/US2024/062216 filed Dec. 29, 2024, all of which are incorporated herein by reference in their entirety.

### FIELD OF THE DISCLOSURE

Cosmetic compositions providing enhanced penetration of actives into skin are provided along with methods of use related thereto.

### BACKGROUND OF THE DISCLOSURE

There have been a wide variety of skin care active ingredients used in the field of cosmetics and dermatological compositions.

It is preferable for skin care active ingredients to be absorbed by the skin or penetrated into the skin as much as possible. However, skin absorption or skin penetration of such skin care active ingredients is not easy due to the barrier function of the skin.

The inventors have overcome these challenges and provide a composition that enhance the skin penetration of a skin care active ingredient by using a skin penetration enhancer with the skin care active ingredient.

### SUMMARY OF THE DISCLOSURE

The instant disclosure relates to cosmetic compositions providing improved penetration of desired skin active agents into adult human skin. The inventors discovered that certain amounts and combinations of ingredients enable cosmetic compositions to provide improved penetration of desired skin active agents without the use of common penetration enhancers.

The instant disclosure relates to cosmetic compositions that include:
(a) at least hydroxyethylurea;
(b) at least three natural moisturizing factors;
(c) at least one N-substituted aminosulfonic acid compound; and
wherein the formula is stable.

In some embodiments, the at least one natural moisturizer factors are selected from the group consisting of glycine, serine, alanine, sodium pca and combination thereof. In various embodiments, the at least one natural moisturizer factors comprise at least three natural moisturizer factors comprising glycine, serine, alanine. In some embodiments, the at least one natural moisturizer factors are present in an amount from about 0.2 to about 9.5 wt. % based on the total weight of the cosmetic composition.

In one or more embodiments, the at least one N-substituted aminosulfonic acid compound is selected from the group consisting of N,N-bis[2-hydroxyethyl]-2-aminoethanesulfonic acid, N-2-hydroxyethylpiperazine-N′-2-ethanesulfonic acid, 3-[N-morpholino]propanesulfonic acid, piperazine-N,N′-bis[2-ethanesulfonic]acid, 3-[N-tris(hydroxymethyl)methylamino]-2-hydroxypropanesulfonic acid, 2-[N-morpholino]ethanesulfonic acid, N-(2-acetamido)-2-aminoethanesulfonic acid, and N-tris(hydroxymethyl)methyl-2-aminoethanesulfonic acid and combination thereof. In various embodiments, the at least one N-substituted aminosulfonic acid compound is present from about 0.15 to about 4.8 wt. % based on the total weight of the cosmetic composition.

In various embodiments, the pH of the composition is from about 4.5 to about 7.

In one or more embodiments, the cosmetic compositions may include fatty compounds.

In one or more embodiments, the cosmetic compositions may include polymers.

In one or more embodiments, the cosmetic compositions may include surfactants.

In one or more embodiments, the cosmetic compositions may include water-soluble solvents.

In various embodiments, the cosmetic compositions may include at least one skin active agent. In some embodiments, the at least one skin active agent selected from antioxidants, free-radical scavengers, moisturizers, bleaching agents, liporegulators, anti-acne agents, antiseborrhoeic agents, anti-ageing agents, softeners, anti-wrinkle agents, anti-inflammatories, refreshing agents, cicatrizing agents, vascular protective agents, antibacterials, antifungals, exfoliating agents, antiperspirants, deodorants, skin conditioners, desensitizing agents, immunemodulators and nourishing agents, moisture absorbers, soothing active ingredients and sebum absorbing ingredients.

In some embodiments, the cosmetic compositions may contain:
(a) about 1.3 to about 25 wt. % of hydroxyethyl urea;
(b) about 0.1 to about 10 wt. % of natural moisturizing factors;
(c) about 0.1 to about 5 wt. % of at least one N-substituted aminosulfonic acid compound; and
wherein the formula is stable and the penetration of hydroxyethyl urea is improved, and all weight percentages are based on the total weight of the cosmetic composition.

In some embodiments, the cosmetic compositions may contain:
(a) from about 1.3 to about 25 wt. % of hydroxyethyl urea;
(b) from about 0.1 to about 10 wt. % of natural moisturizing factors;
(c) from about 0.1 to about 5 wt. % of hydroxyethylpiperazine ethane sulfonic acid; and
wherein the formula is stable and the penetration of the hydroxyethyl urea is improved, and all weight percentages are based on the total weight of the cosmetic composition.

The cosmetic compositions are useful for treating the skin, in particular the skin of the face.

Some aspects of the instant disclosure can include a method for treating the skin comprising applying the cosmetic composition to the skin.

The cosmetic compositions of the instant disclosure provide unexpected, improved penetration of skin active agents without the use of common penetration enhancers.

It is to be understood that both the foregoing general description and the following detailed description are exemplary and explanatory only and are not restrictive of the invention.

### BRIEF DESCRIPTION OF THE DRAWING

This application contains at least one drawing executed in color. Copies of this patent or patent application publication with color drawing(s) will be provided by the Office upon request and payment of the necessary fee.

Features and advantages of the general inventive concepts will become apparent from the following detailed description made with reference to the accompanying drawings.

FIG. 1: ATR-FTIR data showing the distribution of the protein content (skin) on untreated human skin (control) and after topical application of the Products A, B and C (n=3) provided by the sponsor (layer 0) and after sequential tape strips (layer 1, 2, 4, 6, 8 and 10)

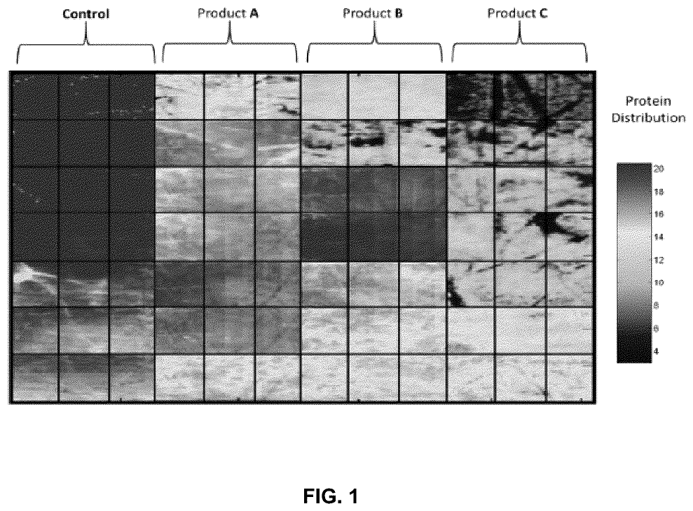

FIG. 2: ATR-FTIR data showing the distribution of the hydroxyethyl urea based on the 1060 cm⁻¹ band area into untreated human skin (control), and skin samples treated with the Products A, B and C.

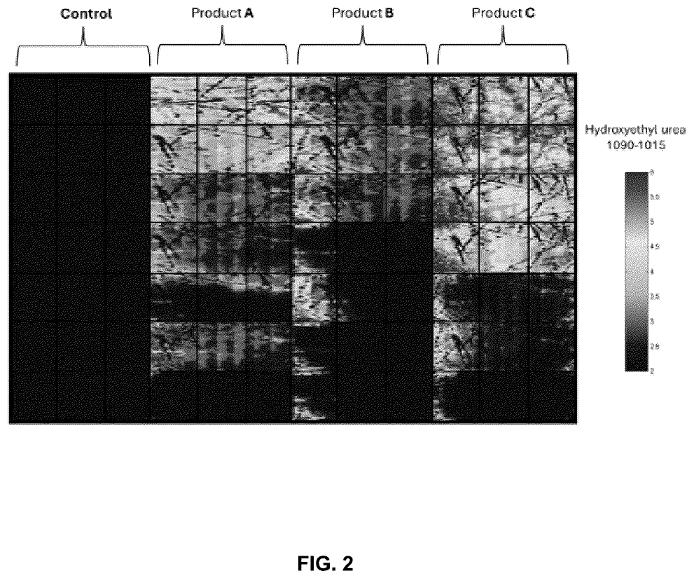

Aspects and many of the attendant advantages of the claimed subject matter will become more readily appreciated as the same become better understood by reference to the following detailed description, when taken in conjunction with the accompanying drawings.

The detailed description set forth below in connection with the appended drawings, where like numerals reference like elements, is intended as a description of various embodiments of the disclosed subject matter and is not intended to represent the only embodiments. The embodiments described in this disclosure are provided merely as examples or illustrations and should not be construed as preferred or advantageous over other embodiments. The illustrative examples provided herein are not intended to be exhaustive or to limit the claimed subject matter to the exact forms disclosed.

### DETAILED DESCRIPTION OF THE DISCLOSURE

The present disclosure relates to cosmetic compositions providing improved penetration of desired skin active agents into adult human skin. As noted above, the inventors discovered that certain amounts and combinations of ingredients enable cosmetic compositions to provide improved penetration of desired skin active agents without the use of common penetration enhancers.

The instant disclosure relates to cosmetic compositions that include:
(a) at least one skin penetration enhancers;
(b) at least three natural moisturizing factors;
(c) at least one N-substituted aminosulfonic acid compound; and
wherein the formula is stable

### Skin Penetration Enhancers

Some therapeutic or cosmetic compositions achieve epidermal penetration by using a skin penetration enhancing carrier or vehicle. Such carrier or vehicles (which are compounds or mixtures of compounds) are often described as “penetration enhancers” or “skin enhancers.” Typical penetration enhancers, which may be optionally excluded from the cosmetic composition, include sulphoxides (such as dimethylsulphoxide, DMSO), azones (e.g., laurocapram), pyrrolidones, (for example 2-pyrrolidone, 2P), alcohols and alkanols (ethanol, or decanol), surfactants (also common in dosage forms) and terpenes. Some penetration enhancers are capable of inserting between the hydrophobic tails of the bilayer, thus disturbing their packing, increasing their fluidity and, subsequently, leading to easier diffusion of lipid-like penetrants. The inventors surprisingly discovered that certain cosmetic compositions according to the disclosure provided improved penetration of skin active agents into adult human skin without necessitating the use of certain penetration enhancers. Nevertheless, in some cases, it may be desirable to include penetration enhancers, such as those discussed herein.

In accordance with the disclosure, provided are cosmetic compositions that include one or more skin penetration enhancers. In exemplary embodiments, the skin penetration enhancers aid in penetration of the cosmetic composition into the skin. In other embodiments, the composition may include any one or more of skin penetration enhancers selected from the group consisting of hydroxyethyl urea Sodium Dilauramidoglutamide Lysine, glycols, dimethyl isosorbide, ethoxydiglycol, polysorbates, PEGs, and combinations thereof. Skin penetration enhancers may include, but are not limited to, hydroxyethyl urea.

The skin penetration enhancers may be provided in the composition in any appropriate amount. More particularly, the total amount of the at least one skin penetration enhancers may be present in the cosmetic composition from about 1.5 to about 24 wt. %. In further embodiments, the cosmetic compositions includes about 1 to about 22 wt. %, about 1 to about 20 wt. %, about 1 to about 18 wt. %, about 1 to about 15 wt. %, about 2 to about 24 wt. %, about 2 to about 22 wt. %, about 2 to about 20 wt. %, about 2 to about 18 wt. %, about 2 to about 15 wt. %, about 4 to about 24 wt. %, about 4 to about 22 wt. %, about 4 to about 20 wt. %, about 4 to about 18 wt. %, about 4 to about 15 wt. %, about 5 to about 24 wt. %, about 5 to about 22 wt. %, about 5 to about 20 wt. %, about 5 to about 18 wt. %, about 5 to about 15 wt. %, about 8 to about 24 wt. %, about 8 to about 22 wt. %, about 8 to about 20 wt. %, or about 8 to about 15 wt. % or any value, range, or sub-range therebetween, of at least one skin penetration enhancers, based on the total weight of the cosmetic composition.

### Natural Moisturizing Factors

The term “Natural Moisturizing Factor” or “NMF” as used herein refers to a group of hygroscopic compounds found within the stratum corneum, the outermost layer of the epidermis. These compounds act as humectants, drawing water from the atmosphere and binding it to the skin's surface. This process is essential for maintaining skin hydration, flexibility, and barrier function.

In accordance with the disclosure, provided are cosmetic compositions that include one or more natural moisturizing factors. In exemplary embodiments, natural moisturizing factors aid in penetration of the cosmetic composition into the skin. In other embodiments, the composition may include any one or more of natural moisturizing factors selected from the group consisting of glycine, alanine, serine, sodium pca and combinations thereof.

Non-limiting examples of natural moisturizing factors that may be mentioned in particular, alone or as a mixture include free amino acids, pyrrolidone carboxylic acid, lactates, sugars, inorganic acids, peptides, chloride, sodium, potassium, creatinine uric acid, glucosamine, calcium, magnesium, citrate, formate, phosphate.

The natural moisturizing factors may be provided in the composition in any appropriate amount. More particularly, the total amount of the at least one natural moisturizing factor may be present in the cosmetic composition from about 0.1 to about 10 wt. %. In further embodiments, the cosmetic compositions includes about 0.1 to about 9.5 wt. %, about 0.1 to about 9.0 wt. %, about 0.1 to about 8.5 wt. %, about 0.1 to about 8.0 wt. %, about 0.2 to about 10 wt. %, about 0.22 to about 9.5 wt. %, about 0.2 to about 9.0 wt. %, about 0.2 to about 8.5 wt. %, about 0.2 to about 8.0 wt. %, about 0.3 to about 10 wt. %, about 0.3 to about 9.5 wt. %, about 0.3 to about 9.0 wt. %, about 0.3 to about 8.5 wt. %, about 0.3 to about 8.0 wt. %, about 0.4 to about 10 wt. %, about 0.4 to about 9.5 wt. %, about 0.5 to about 9.0 wt. %, about 0.5 to about 8.5 wt. %, about 0.5 to about 8.0 wt. %, about 1.0 to about 10 wt. %, about 1 to about 9.0 wt. %, about 1.0 to about 8.0 wt. %, or about 1.0 to about 7 wt. % or any value, range, or sub-range therebetween, of at least one natural moisturizing factors, based on the total weight of the cosmetic composition.

### N-Substituted Aminosulfonic Acid Compounds

In accordance with the various embodiments, the composition according to the instant disclosure comprises at least one N-substituted aminosulfonic acid compound.

Suitable N-substituted aminosulfonic acid compounds include, but are not limited to, N,N-bis[2-hydroxyethyl]-2-aminoethanesulfonic acid, N-2-hydroxyethylpiperazine-N′-2-ethanesulfonic acid, 3-[N-morpholino]propanesulfonic acid, piperazine-N,N′-bis[2-ethanesulfonic]acid, 3-[N-tris(hydroxymethyl)methylamino]-2-hydroxypropanesulfonic acid, 2-[N-morpholino]ethanesulfonic acid, N-(2-acetamido)-2-aminoethanesulfonic acid, and N-tris(hydroxymethyl)methyl-2-aminoethanesulfonic acid. A mixture of these acids may also be used.

Preferably, the N-substituted aminosulfonic acid compound is selected from N-2-hydroxyethylpiperazine-N′-2-ethanesulfonic acid, piperazine-N,N′-bis[2-ethanesulfonic]acid, and a combination thereof. More preferably, the N-substituted aminosulfonic acid compound is N-2-hydroxyethylpiperazine-N′-2-ethanesulfonic acid, also called hydroxylethylpiperzine ethanesulfonic acid.

The inventors have found that using N-substituted aminosulfonic acid compounds, in particular, hydroxylethylpiperzine ethanesulfonic acid can improve the stability in terms of minimizing pH shifts and helps the penetration of hydroxyethyl urea.

As a commercial product of N-substituted aminosulfonic acid compounds, mention can be made of hydroxylethylpiperzine ethane sulfonic acid sold under the name HEPES-LUV by the company TAIWAN HOPAX.

The at least one N-substituted aminosulfonic acid compound may be provided in the composition in any appropriate amount. More particularly, the total amount of the at least one N-substituted aminosulfonic acid compound may be present in the cosmetic composition from about 0.1 to about 5.0 wt. %. In further embodiments, the cosmetic compositions includes about 0.1 to about 4.8 wt. %, about 0.1 to about 4.6 wt. %, about 0.1 to about 4.4 wt. %, about 0.1 to about 4.2 wt. %, about 0.1 to about 4.0 wt. %, about 0.2 to about 5.0 wt. %, 0.2 to about 4.8 wt. %, 0.2 to about 4.6 wt. %, 0.2 to about 4.4 wt. %, 0.2 to about 4.2 wt. %, 0.2 to about 4.0 wt. %, about 0.3 to about 5.0 wt. %, about 0.3 to about 4.8 wt. %, about 0.3 to about 4.6 wt. %, about 0.3 to about 4.4 wt. %, about 0.3 to about 4.2 wt. %, about 0.3 to about 4.0 wt. %, about 0.4 to about 5.0 wt. %, about 0.4 to about 4.8 wt. %, .%, about 0.4 to about 4.6 wt. %, .%, about 0.4 to about 4.4 wt. %, .%, about 0.4 to about 4.2 wt. %, about 0.4 to about 4.0 wt. %, about 0.5 to about 5.0 wt. %, about 0.5 to about 4.8 wt. %, about 0.5 to about 4.6 wt. %, about 0.5 to about 4.4 wt. %, about 0.5 to about 4.2 wt. %, about 0.5 to about 4.0 wt. %, about 1.0 to about 5.0 wt. %, about 1.0 to about 4.8 wt. %, about 1.0 to about 4.6 wt. %, about 1.0 to about 4.4 wt. %, about 1.0 to about 4.2 wt. %, about 1.0 to about 4.0 wt. % or any value, range, or sub-range therebetween, of at least one N-substituted aminosulfonic acid compound, based on the total weight of the cosmetic composition.

### Fatty Compounds

Examples of fatty compound(s) that may be incorporated into the skin treatment composition include fatty alcohol, a fatty ester, a fatty ether, a fatty acid, a wax, an oil, a derivative thereof, or a mixture thereof. Additional examples of fatty compounds that are worth mentioning include oils, mineral oil, alkanes (paraffins), fatty alcohol derivatives, fatty acid derivatives, esters of fatty alcohols, hydroxy-substituted fatty acids, waxes, triglyceride compounds, lanolin, and a mixture thereof. One or more fatty compounds(s) may be included in the skin treatment composition has an emulsifier. For example, the fatty compound may be a fatty alcohol that is capable of or is used for emulsifying another ingredient. Although not specifically identified, some of the fatty compounds listed below may be utilized as emulsifiers. Further examples of fatty compounds are discussed below.

### (i) Fatty Ester(s)

The skin treatment compositions may include one or more fatty compound(s) that is a fatty ester. For example, the fatty compound(s) may be chosen from from dialkyl carbonates of formula: R₁O(C═O)R₂, wherein R₁ and R₂ are independently linear or branched, saturated or unsaturated alkyl chains having 1 to 30 carbon atoms, or having 2 to 28 carbon atoms, or having 4 to 25 carbon atoms, or having 6 to 22 carbon atoms, preferably one or more fatty carbonates selected from C14-15 dialkyl carbonate, dicaprylyl carbonate, diethyl carbonate, dihexyl carbonate, diethylhexyl carbonate, dimethoxyphenyl phenyloxoethyl ethylcarbonate, dimethyl carbonate, dipropyl carbonate, dipropylheptyl carbonate, dioctyl carbonate, and a mixture thereof.

Additionally or alternatively, the fatty ester chosen from cetyl ester, purcellin oil (cetearyl octanoate), isopropyl myristate, isopropyl palmitate, C₁₂-C₁₅ alkyl benzoate, 2-ethylphenyl benzoate, isopropyl lanolate, hexyl laurate, diisopropyl adipate, isononyl isononanoate, oleyl erucate, 2-ethylhexyl palmitate, isostearyl isostearate, diisopropyl sebacate, octanoates, decanoates or ricinoleates of alcohols or polyalcohols, hydroxylated esters, dicaprylyl carbonate, pentaerythritol esters, and a mixture thereof. Other fatty esters worth mentioning include polyglyceryl-10 oleate, polyglyceryl-10 dioleate, polyglyceryl-6 stearate, polyglyceryl-6 distearate, polyglyceryl-10 stearate, polyglyceryl-10 distearate, polyglyceryl-8 dipalmitate, polyglyceryl-10 dipalmitate, polyglyceryl-10 behenate, and polyglyceryl-12 trilaurate.

### (ii) Fatty Alcohol(s)

Suitable fatty alcohols, if present, include those having a fatty group with a carbon chain of greater than 8 carbon atoms, 8 to 50 carbon atoms, 8 to 40 carbon atoms, 8 to 30 carbon atoms, 8 to 22 carbon atoms, 12 to 22 carbon atoms, or 12 to 18 carbon atoms, including all ranges and subranges therebetween. In some instances, the fatty group of the fatty alcohols has a carbon chain of 10 to 20 carbon atoms or 10 to 18 carbon atoms. The fatty alcohols may be chosen from polyethylene glycol ethers, such as those having a fatty alcohol group with a carbon chain of 12 to 16 or 12 to 14 carbon atoms.

The fatty alcohol portion is preferably hydrogenated (for example, stearyl, lauryl, cetyl, cetearyl); however, the fatty alcohol may contain one or more double bonds (for example, oleyl). Non-limiting examples of fatty alcohols include decyl alcohol, undecyl alcohol, dodecyl alcohol, myristyl alcohol, lauryl alcohol, cetyl alcohol, stearyl alcohol, cetearyl alcohol (cetyl alcohol and stearyl alcohol), isostearyl alcohol, isocetyl alcohol, behenyl alcohol, linalool, oleyl alcohol, cis-4-t-butylcyclohexanol, isotridecyl alcohol, myricyl alcohol, and a mixture thereof. In some cases, the fatty alcohols comprise at least one of or may be chosen from myristyl alcohol, lauryl alcohol, cetyl alcohol, stearyl alcohol, cetearyl alcohol, isostearyl alcohol, oleyl alcohol, isotridecyl alcohol, and a mixture thereof.

The fatty alcohol may be saturated or unsaturated. Exemplary saturated liquid fatty alcohols may be branched and optionally contain in their structure at least one aromatic or non-aromatic ring. In some instances, however, the fatty alcohols are acyclic. Non-limiting examples of liquid saturated fatty alcohols include octyldodecanol, isostearyl alcohol, and 2-hexyldecanol.

Exemplary unsaturated liquid fatty alcohol may include in their structure at least one double or triple bond. For example, the fatty alcohols may include several double bonds (such as 2 or 3 double bond), which may be conjugated or non-conjugated. The unsaturated fatty alcohols can be linear or branched and may be acyclic or include in their structure at least one aromatic or non-aromatic ring. Liquid unsaturated fatty alcohols may include or be chosen from oleyl alcohol, linoleyl alcohol, linolenyl alcohol and undecylenyl alcohol.

The fatty alcohols may be alkoxylated fatty alcohols, e.g., having about 1 to about 100 moles of an alkylene oxide per mole of alkoxylated fatty alcohol. For example, the alkoxylated fatty alcohols may be alkoxylated with about 1 to about 80 moles, about 2 to about 50, about 5 to about 45 moles, about 10 to about 40 moles, or 15 to about 35 mores, including all ranges and subranges therebetween, of an alkylene oxide per mole of alkoxylated fatty alcohol.

As examples of alkoxylated fatty alcohols, steareth (for example, steareth-2, steareth-20, and steareth-21), laureth (for example, laureth-4, and laureth-12), ceteth (for example, ceteth-10 and ceteth-20) and ceteareth (for example, ceteareth-2, ceteareth-10, and ceteareth-20) are mentioned. In at least one instance, the one or more alkoxylated fatty alcohols include steareth-20. In some instances, the one or more alkoxylated fatty alcohols may be exclusively steareth-20.

Additional fatty alcohol derivatives that may, optionally be suitable include methyl stearyl ether; 2-ethylhexyl dodecyl ether; stearyl acetate; cetyl propionate; the ceteth series of compounds, such as ceteth-1 through ceteth-45, which are ethylene glycol ethers of cetyl alcochol, wherein the numeric designation indicates the number of ethylene glycol moieties present; the steareth series of compounds such as steareth-1 through 10, which are ethylene glycol ethers of steareth alcohol, wherein the numeric designation indicates the number of ethylene glycol moieties present; ceteareth 1 through ceteareth-10, which are the ethylene glycol ethers of ceteareth alcohol, i.e. a mixture of fatty alcohols containing predominantly cetyl and stearyl alcohol, wherein the numeric designation indicates the number of ethylene glycol moieties present; C1-C30 alkyl ethers of the ceteth, steareth, and ceteareth compounds just described; polyoxyethylene ethers of branched alcohols such as octyldodecyl alcohol, dodecylpentadecyl alcohol, hexyldecyl alcohol, and isostearyl alcohol; polyoxyethylene ethers of behenyl alcohol; PPG ethers such as PPG-9-steareth-3, PPG-11 stearyl ether, PPG8-ceteth-1, and PPG-10 cetyl ether; and a mixture thereof.

(iii) Fatty Ether(s)

The fatty compounds may be chosen from fatty ethers. For example, the skin treatment composition may include olyoxyethylene cetyl/stearyl ether, polyoxyethylene cholesterol ether, polyoxyethylene laurate or dilaurate, polyoxyethylene stearate or distearate, polyoxyethylene lauryl or stearyl ether, dicaprylyl ether, dicetyl ether distearyl ether, or a mixture thereof. Non-limiting examples of suitable polyoxyethylene fatty ethers include, but are not limited to, polyoxyethylene cetyl/stearyl ether, polyoxyethylene cholesterol ether, polyoxyethylene laurate or dilaurate, polyoxyethylene stearate or distearate, polyoxyethylene lauryl or stearyl ether, and mixtures thereof, wherein the polyoxyethylene head group ranges from about 2 to about 100 groups. In certain embodiments, the polyoxyethylene fatty ethers include polyoxyethylene stearyl ether, polyoxyethylene myristyl ether, polyoxyethylene lauryl ether having from about 3 to about 10 oxyethylene units and mixtures thereof.

### (iv) Fatty Acid(s)

In some instances, the fatty compounds may be chosen from fatty acids, fatty acid derivatives, esters of fatty acids, hydroxyl-substituted fatty acids, and alkoxylated fatty acids. The fatty acids may be straight or branched chain acids and/or may be saturated or unsaturated. Non-limiting examples of fatty acids include diacids, triacids, and other multiple acids as well as salts of these fatty acids. For example, the fatty acid may optionally include or be chosen from lauric acid, palmitic acid, stearic acid, behenic acid, arichidonic acid, oleic acid, isostearic acid, sebacic acid, and a mixture thereof. In some cases, the fatty acids are selected from the group consisting of palmitic acid, stearic acid, and a mixture thereof.

Non-limiting examples of polyglycerol esters of fatty acids include those of the following formula:

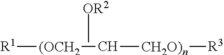

wherein the average value of n is about 3 and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C₁-C₄₀, C₁-C₃₀, C₁-C₂₅, or C₁-C₂₀, C₁-C₁₆, or C₁-C₁₀.

The fatty acid derivatives are defined herein to include fatty acid esters of the fatty alcohols as defined above, fatty acid esters of the fatty alcohol derivatives as defined above when such fatty alcohol derivatives have an esterifiable hydroxyl group, fatty acid esters of alcohols other than the fatty alcohols and the fatty alcohol derivatives described above, hydroxy-substituted fatty acids, and a mixture thereof. Non-limiting examples of fatty acid derivatives include ricinoleic acid, glycerol monostearate, 12-hydroxy stearic acid, ethyl stearate, cetyl stearate, cetyl palmitate, polyoxyethylene cetyl ether stearate, polyoxyethylene stearyl ether stearate, polyoxyethylene lauryl ether stearate, ethyleneglycol monostearate, polyoxyethylene monostearate, polyoxyethylene distearate, propyleneglycol monostearate, propyleneglycol distearate, trimethylolpropane distearate, sorbitan stearate, sorbitan tristearate, polyglyceryl stearate, dimethyl sebacate, PEG-15 cocoate, PPG-15 stearate, glyceryl monostearate, glyceryl distearate, glyceryl tristearate, PEG-8 laurate, PPG-2 isostearate, PPG-9 laurate, and a mixture thereof. Preferred for use herein are glycerol monostearate, 12-hydroxy stearic acid, and a mixture thereof.

### (v) Wax(es)

The fatty compounds may, in some instances, include or be chosen from one or more waxes. Non-limiting examples of waxes in this category include for example, synthetic wax, ceresin, paraffin, ozokerite, polyethylene waxes, illipe butter, beeswax, carnauba, microcrystalline, lanolin, lanolin derivatives, candelilla, cocoa butter, shellac wax, spermaceti, bran wax, capok wax, sugar cane wax, montan wax, whale wax, bayberry wax, acacia decurrents flower wax, vegetable waxes (such as sunflower seed (Helianthus annuus), carnauba, candelilla, ouricury or japan wax or cork fibre or sugarcane waxes), or a mixture thereof.

### (vi) Oil(s)

The skin treatment compositions include one or more oils. The oil component of the NLCs is typically has melting temperature of less than 45° C., a molecular weight of at least 190, and a solubility in water of no greater than 1 part in 99 parts of water.

Non-limiting examples of include, but are not limited to, natural oils, such as coconut oil; hydrocarbons, such as mineral oil and hydrogenated polyisobutene; esters, such as C₁₂-C₁₅ alkyl benzoate; diesters, such as propylene dipelarganate; and triesters, such as glyceryl trioctanoate. Further examples of oils that may, optionally, be included in the skin treatment compositions include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco-dicaprylate/caprate, decyl isostearate, isodecyl oleate, isodecyl neopentanoate, isohexyl neopentanoate, octyl palmitate, dioctyl malate, tridecyl octanoate, myristyl myristate, octododecanol, or combinations of octyldodecanol, acetylated lanolin alcohol, cetyl acetate, isododecanol, polyglyceryl-3-diisostearate, castor oil, lanolin and lanolin derivatives, triisocetyl citrate, sorbitan sesquioleate, C₁₀-C₁₈ triglycerides, caprylic/capric/triglycerides, coconut oil, corn oil, cottonseed oil, glyceryl triacetyl hydroxystearate, glyceryl triacetyl ricinoleate, glyceryl trioctanoate, hydrogenated castor oil, linseed oil, mink oil, olive oil, palm oil, illipe butter, rapeseed oil, soybean oil, sunflower seed oil, tallow, tricaprin, trihydroxystearin, triisostearin, trilaurin, trilinolein, trimyristin, triolein, tripalmitin, tristearin, walnut oil, wheat germ oil, cholesterol, or combinations thereof.

Additionally, or alternatively, the oil may be selected from plant based and/or vegetable oils. Non-limiting examples of plant-based or vegetable oils include acai oil, almond oil, aloe vera oil, andiroba oil, annatto oil, avocado oil, babassu oil, borage oil, brazil nut oil, buriti oil, camelina oil, coffee oil, copaiba oil, emu oil, passion fruit oil, almond oil, Ricinus communis (castor) seed oil, coconut oil, grapeseed oil, jojoba oil, macadamia nut oil, rose hip oil, ajwain oil, angelic root oil, anise oil, aragan oil, asafetida, balsam oil, basil oil, bay oil, bergamot oil, black pepper essential oil, buchu oil, birch oil, camphor, cannabis oil, caraway oil, cardamom seed oil, carrot seed oil, chamomile oil, calamus root oil, cinnamon oil, citronella oil, clary sage, clove leaf oil, coffee, coriander, costmary oil, cranberry seed oil, cubeb, cumin oil, cypress, cypriol, curry leaf, davana oil, dill oil, elecampane, eucalyptus oil, fennel seed oil, fenugreek oil, fir, frankincense oil, galangal, geranium oil, ginger oil, goldenrod, grapefruit oil, grapeseed oil, henna oil, helichrysum, horseradish oil, hyssop, Idaho tansy, jasmine oil, juniper berry oil, lavender oil, lemon oil, lemongrass, marjoram, melaleuca, lemon balm oil, mountain savory, mugwort oil, mustard oil, myrrh oil, myrtle, neem tree oil, neroli, nutmeg, orange oil, oregano oil, orris oil, palo santo, parsley oil, patchouli oil, perilla oil, pennyroyal oil, peppermint oil, petitgrain, pine oil, plum oil, ravensara, red cedar, roman chamomile, rose oil, rosehip oil, rosemary oil, rosewood oil, sandalwood oil, sassafras oil, savory oil, schisandra oil, spikenard, spruce, star anise oil, tangerine, tarragon oil, tea tree oil, thyme oil, tsuga oil, turmeric, valerian, vetiver oil, western red cedar, wintergreen, yarrow oil, ylang-ylang, and zedoary oil.

Non-limiting examples of liquid triglycerides and oils of plant origin include alexandria laurel tree oil, avocado oil, apricot stone oil, barley oil, borage seed oil, calendula oil, canelle nut tree oil, canola oil, caprylic/capric triglyceride castor oil, coconut oil, corn oil, cotton oil, cottonseed oil, evening primrose oil, flaxseed oil, groundnut oil, hazelnut oil, glycereth triacetate, glycerol triheptanoate, glyceryl trioctanoate, glyceryl triundecanoate, hempseed oil, jojoba oil, lucerne oil, maize germ oil, marrow oil, millet oil, neopentylglycol dicaprylate/dicaprate, olive oil, palm oil, passionflower oil, pentaerythrityl tetrastearate, poppy oil, propylene glycol ricinoleate, rapeseed oil, rye oil, safflower oil, sesame oil, shea butter, soya oil, soybean oil, sweet almond oil, sunflower oil, sysymbrium oil, syzigium aromaticum oil, tea tree oil, walnut oil, wheat germ glycerides and wheat germ oil.

### Thickening Agent(s)

The skin treatment compositions described herein may, optionally, include a thickening agent. The amount of thickening agents can vary but is typically from about 0.01 to about 20 wt. %, based on the total weight of the skin treatment composition. In some instances, the amount of fatty compounds present in the skin treatment compositions is about 0.1 to 20 wt. %, about 0.1 to about 18 wt. %, about 0.1 to about 16 wt. %, about 0.1 to about 14 wt. %, about 0.1 to about 12 wt. %, about 0.1 to about 10 wt. %, about 0.1 to about 8 wt. %, about 0.1 to about 7 wt. %, about 0.1 to about 6 wt. %, about 0.1 to about 5 wt. %; about 0.5 to 20 wt. %, about 0.5 to about 18 wt. %, about 0.5 to about 16 wt. %, about 0.5 to about 14 wt. %, about 0.5 to about 12 wt. %, about 0.5 to about 10 wt. %, about 0.5 to about 8 wt. %, about 0.5 to about 7 wt. %, about 0.5 to about 6 wt. %, about 0.5 to about 5 wt. %; about 1 to about 20 wt. %, about 1 to about 18 wt. %, about 1 to about 16 wt. %, about 1 to about 14 wt. %, about 1 to about 12 wt. %, about 1 to about 10 wt. %, about 1 to about 8 wt. %, about 1 to about 7 wt. %, about 1 to about 6 wt. %, about 1 to about 5 wt. %; about 2 to about 20 wt. %, about 2 to about 18 wt. %, about 2 to about 16 wt. %, about 2 to about 14 wt. %, about 2 to about 12 wt. %, about 2 to about 10 wt. %, about 2 to about 8 wt. %, about 2 to about 7 wt. %, about 2 to about 6 wt. %, about 2 to about 5 wt. %; about 3 to about 20 wt. %, about 3 to about 18 wt. %, about 3 to about 16 wt. %, about 3 to about 14 wt. %, about 3 to about 12 wt. %, about 3 to about 10 wt. %, about 3 to about 8 wt. %, about 3 to about 7 wt. %, about 3 to about 6 wt. %, about 3 to about 5 wt. %; about 4 to about 20 wt. %, about 4 to about 18 wt. %, about 4 to about 16 wt. %, about 4 to about 14 wt. %, about 4 to about 12 wt. %, about 4 to about 10 wt. %, about 4 to about 8 wt. % about 4 to about 7 wt. %, about 4 to about 6 wt. %, about 4 to about 5 wt. %; about 5 to about 20 wt. %, about 5 to about 18 wt. %, about 5 to about 16 wt. %, about 5 to about 14 wt. %, about 5 to about 12 wt. %, about 5 to about 10 wt. %, or about 5 to about 8 wt. %, about 5 to about 7 wt. %, or about 5 to about 6 wt. %, including all ranges and sub-ranges therebetween, based on the total weight of the skin treatment composition.

The thickening agent(s) may be chosen from xanthan gum, guar gum, biosaccharide gum, cellulose, acacia seneca gum, sclerotium gum, agarose, pechtin, gellan gum, hyaluronic acid. Additionally, the one or more thickening agents may include polymeric thickening agents selected from the group consisting of ammonium polyacryloyldimethyl taurate, ammonium acryloyldimethyltaurate/VP copolymer, sodium polyacrylate, acrylates copolymers, polyacrylamide, carbomer, and acrylates/C10-30 alkyl acrylate crosspolymer. In some cases, the skin treatment composition includes ammonium polyacryloyldimethyl taurate and/or sodium polyacrylate. Suitable thickening agents may be found in U.S. patent application Ser. No. 16/731,654, which is incorporated herein, in its entirety for all purposes.

Many thickening agents are water-soluble and increase the viscosity of water or form an aqueous gel when the skin treatment composition of the invention is dispersed/dissolved in water. The aqueous solution may be heated and cooled, or neutralized, for forming the gel, if necessary. The thickener may be dispersed/dissolved in an aqueous solvent that is soluble in water, e.g., ethyl alcohol when it is dispersed/dissolved in water.

Particular types of thickening agents that may be mentioned include the following:

One or more thickening agents can optionally be included in the skin treatment compositions of the instant disclosure. Thickening agents may be referred to as “thickeners” or “viscosity modifying agents.” Thickening agents are typically included to increase the viscosity of the skin treatment compositions. Nonetheless, in some instances, certain thickening agents provide additional, surprising benefits to the skin treatment compositions. Non-limiting examples of thickening agents include polyacrylate crosspolymers or crosslinked polyacrylate polymers, cationic acrylate copolymers, anionic acrylic or carboxylic acid polymers, polyacrylamide polymers, polysaccharides such as cellulose derivatives, gums, polyquaterniums, vinylpyrrolidone homopolymers/copolymers, C8-24 hydroxyl substituted aliphatic acid, C8-24 conjugated aliphatic acid, sugar fatty esters, polyglyceryl esters, and a mixture thereof. Particular types of thickening agents that may be mentioned include the following:

Carboxylic Acid or Carboxylate Based Homopolymer or Co-Polymer, which can be Linear or Crosslinked:

These polymers contain one or more monomers derived from acrylic acid, substituted acrylic acids, and salts and esters of these acrylic acids (acrylates) and the substituted acrylic acids. Commercially available polymers include those sold under the trade names CARBOPOL, ACRYSOL, POLYGEL, SOKALAN, CARBOPOL ULTREZ, and POLYGEL. Examples of commercially available carboxylic acid polymers include the carbomers, which are homopolymers of acrylic acid crosslinked with allyl ethers of sucrose or pentaerytritol. The carbomers are available as the CARBOPOL 900 series from B.F. Goodrich (e.g., CARBOPOL 954). In addition, other suitable carboxylic acid polymeric agents include ULTREZ 10 (B.F. Goodrich) and copolymers of C10-30 alkyl acrylates with one or more monomers of acrylic acid, methacrylic acid, or one of their short chain (i.e., C1-4 alcohol) esters, wherein the crosslinking agent is an allyl ether of sucrose or pentaerytritol. These copolymers are known as acrylates/C10-C30 alkyl acrylate crosspolymers and are commercially available as CARBOPOL 1342, CARBOPOL 1382, PEMULEN TR-1, and PEMULEN TR-2, from B.F. Goodrich.

Other suitable carboxylic acid or carboxylate polymeric agents include copolymers of acrylic acid and alkyl C5-C10 acrylate, copolymers of acrylic acid and maleic anhydride, and polyacrylate crosspolymer-6. Polyacrylate Crosspolymer-6 is aviable in the raw material known as SEPIMAX ZEN from Seppic.

Another suitable carboxylic acid or carboxylate polymeric agent includes acrylamidopropyltrimonium chloride/acrylates copolymer, a cationic acrylates copolymer (or a quaternary ammonium compound), available as a raw maerial known under the tradename of SIMULQUAT HC 305 from Seppic.

In certain embodiments, the carboxylic acid or carboxylate polymer thickening agents useful herein are those selected from carbomers, acrylates/C10-C30 alkyl acrylate crosspolymers, polyacrylate crosspolymer-6, acrylamidopropyltrimonium chloride/acrylates copolymer, and mixtures thereof.

### Celluloses:

Non-limiting examples of celluloses include cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. In some instances, the cellulose is selected from water-soluble cellulose derivatives (for example, carboxymethyl cellulose, methyl cellulose, methylhydroxypropyl cellulose, hydroxyethyl cellulose, hydroxypropyl cellulose, cellulose sulfate sodium salt). Furthermore, in some instance, the cellulose is preferably hydroxypropylcellulose (HPC).

### Polyvinylpyrrolidone (PVP) and Co-Polymers:

Non-limiting examples include Polyvinylpyrrolidone (PVP), Polyvinylpyrrolidone (PVP)/vinyl acetate copolymer (PVP/VA copolymer), polyvinylpyrrolidone (PVP)/eicosene copolymer, PVP/hexadecene copolymer, etc. Commercially available polyvinylpyrrolidone includes LUVISKOL K30, K85, K90 available from BASF. Commerically available copolymers of vinylpyrrolidone and vinylacetate include LUVISKOL VA37, VA64 available from BASF; copolymers of vinylpyrrolidone, methacrylamide, and vinylimidazole (INCI: VP/Methacrylamide/Vinyl Imidazole Copolymer) is commercially available as LUVISET from BASF. In some instances, PVP and PVP/VA copolymer are preferred.

### Sucrose Esters:

Non-limiting examples include sucrose palmitate, sucrose cocoate, sucrose monooctanoate, sucrose monodecanoate, sucrose mono- or dilaurate, sucrose monomyristate, sucrose mono- or dipalmitate, sucrose mono- and distearate, sucrose mono-, di- or trioleate, sucrose mono- or dilinoleate, sucrose pentaoleate, sucrose hexaoleate, sucrose heptaoleate or sucrose octooleate, and mixed esters, such as sucrose palmitate/stearate, and mixtures thereof.

### Polyglyceryl Esters:

Non-limiting polyglycerol esters of fatty acids (polygylceryl esters) include those of the following formula:

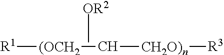

wherein n is from 2 to 20 or from 2 to 10 or from 2 to 5, or is 2, 3, 4, 5, 6, 7, 8, 9, or 10, and R¹, R² and R³ each may independently be a fatty acid moiety or hydrogen, provided that at least one of R¹, R², and R³ is a fatty acid moiety. For instance, R¹, R² and R³ may be saturated or unsaturated, straight or branched, and have a length of C₁-C₄₀, C₁-C₃₀, C₁-C₂₅, or C₁-C₂₀, C₁-C₁₆, or C₁-C₁₀. Additionally, non-limiting examples of nonionic polyglycerol esters of fatty acids include polyglyceryl-4 caprylate/caprate, polyglyceryl-10 caprylate/caprate, polyglyceryl-4 caprate, polyglyceryl-10 caprate, polyglyceryl-4 laurate, polyglyceryl-5 laurate, polyglyceryl-6 laurate, polyglyceryl-10 laurate, polyglyceryl-10 cocoate, polyglyceryl-10 myristate, polyglyceryl-10 oleate, polyglyceryl-10 stearate, and mixtures thereof.

### Gums:

Non-limiting examples of gums include gum arabic, tragacanth gum, karaya gum, guar gum, gellan gum, tara gum, locust bean gum, tamarind gum, xanthan gum, locust bean gum, Seneca gum, sclerotium gum, gellan gum, etc.

### Emulsifiers

The cosmetic composition includes one or more emulsifiers. The emulsifiers include polymeric emulsifiers and non-polymeric emulsifiers. Furthermore, the emulsifiers may be nonionic, anionic, amphoteric, or a combination thereof. Cationic emulsifiers may also be useful but generally, nonionic, and anionic emulsifiers are preferred.

In a preferred embodiment, the cosmetic composition includes one or more polymeric emulsifiers, one or more nonionic emulsifiers, or a combination thereof.

Nonlimiting examples of polymeric emulsifiers include poloxamers, alkoxylated surfactants, polyacrylamide, acryloyldimethyltaurate polymers, or combinations thereof. As the name indicates, polymeric emulsifiers provide emulsifying properties to the cosmetic composition. Nonetheless, in some cases, in addition to their emulsifying properties, the polymeric emulsifiers have additional influence other characteristics of the cosmetic composition, for example, the polymeric emulsifiers can thicken the cosmetic composition, i.e., behave as a thickening agent by increasing the viscosity of the cosmetic composition. In some cases, a polymeric emulsifier may have film forming properties in addition to its emulsifying properties. In instances where a polymeric emulsifier also provides thickening or behaves as a film forming polymer, a single compound (a single polymer/copolymer) will serve as only a polymeric emulsifier or film-forming polymer. A single compound (a single polymer/copolymer) cannot simultaneously serve as both the polymeric emulsifier and the film-forming polymer when the cosmetic composition includes both a polymeric emulsifier and a film-forming polymer.

Poloxamers are nonionic triblock copolymers composed of a central hydrophobic chain of polyoxypropylene (poly(propylene oxide)) flanked by two hydrophilic chains of polyoxyethylene (poly(ethylene oxide)). The term poloxamer emulsifiers refers to a polyethylene oxide-polypropylene oxide-polyethylene oxide (PEG-PPG-PEG) nonionic triblock copolymer. Poloxamer emulsifiers include nonionic triblock copolymers such as polyoxyethylene oxide-polyoxypropylene oxide-polyoxyethylene oxide (PEO-PPO-PEO) characterized by a central hydrophobic chain of polyoxypropylene (poly(propylene oxide)) flanked by two hydrophilic chains of polyoxyethylene (poly(ethylene oxide)). Exemplary nonionic triblock copolymers may comprise a structure according to Formula (A):

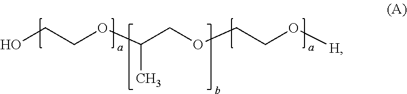

wherein each a is independently an integer in the range of 2-130, and b is an integer in the range of 15-67. In some embodiments, a is in the range of 50-100 and b is in the range of 20-40. In some embodiments, a is in the range of 70-90 and b is in the range of 25-30. Nonlimiting examples of poloxamers include poloxamer 188, also known as Pluronic® F-68, or KOLLIPHOR® P188, e.g., having a=80 and b=27. Other poloxamers include poloxamer 338, also known as Synperonic® PE/F108, poloxamer 407, also known as Synperonic® PE/F127, poloxamer 331, also known as Synperonic® PE/L101.

The term “PLURONIC® F68,” “Pluronic F-68”, or “PF-68”, also known as poloxamer 188, refers to poly(ethylene glycol)-block-poly(propylene glycol)-block poly(ethylene glycol) copolymer with an average molecular weight, avg. Mn, of 8350-8400. The term “PLURONIC® F127” also known as poloxamer 407 refers to a triblock copolymer consisting of a central hydrophobic block of polypropylene glycol flanked by two hydrophilic blocks of polyethylene glycol (PEG). The approximate lengths of the two PEG blocks is 101 repeat units, while the approximate length of the propylene glycol block is 56 repeat units. This is also known by the Croda trade name Synperonic® PE/F 127. The term “PLURONIC® F108” refers to poly(ethylene glycol)-block-poly(propylene glycol)-block poly(ethylene glycol). The term “PLURONIC® P103” refers to poly(ethylene glycol)-block-poly(propylene glycol)-block poly(ethylene glycol). The term “PLURONIC® P104” refers to poly(ethylene glycol)-block-poly(propylene glycol)-block poly(ethylene glycol). The term “PLURONIC® P123” refers to poly(ethylene glycol)-block-poly(propylene glycol)-block poly(ethylene glycol).

Nonlimiting examples of poloxamers include poloxamer-101, poloxamer-105, poloxamer-105 benzoate, poloxamer-108, poloxamer-122, poloxamer-123, poloxamer-124, poloxamer-181, poloxamer-182, poloxamer-182 dibenzoate, poloxamer-183, poloxamer-184, poloxamer-185, poloxamer-188, poloxamer-212, poloxamer-215, poloxamer-217, poloxamer-231, poloxamer-234, poloxamer-235, poloxamer-237, poloxamer-238, poloxamer-282, poloxamer-284, poloxamer-288, poloxamer-331, poloxamer-333, poloxamer-334, poloxamer-335, poloxamer-338, poloxamer-401, poloxamer-402, poloxamer-403, and poloxamer-407.

The lengths of the polymer blocks can be customized and therefore, many different poloxamers exist that have slightly different properties. Poloxamer copolymers are commonly named with the letter “P” (for poloxamer) followed by three digits, the first two digits×100 give the approximate molecular mass of the polyoxypropylene core, and the last digit×10 gives the percentage polyoxyethylene content (e.g., P407=Poloxamer with a polyoxypropylene molecular mass of 4,000 g/mol and a 70% polyoxyethylene content). For the Pluronic® and Synperonic® poloxamer tradenames, coding of these copolymers starts with a letter to define its physical form at room temperature (L=liquid, P=paste, F=flake (solid)) followed by two or three digits. The first digit (two digits in a three-digit number) in the numerical designation, multiplied by 300, indicates the approximate molecular weight of the hydrophobic chain; and the last digit×10 gives the percentage polyoxyethylene content (e.g., F-68 indicates a polyoxypropylene molecular mass of 1,800 g/mol and a 80% polyoxyethylene content). An exemplary poloxamer is Pluronic F-68@. PF-68 is a nonionic triblock copolymer polyoxyethylene oxide-polyoxypropylene oxide-polyoxyethylene oxide (PEO-PPO-PEO). Commercially available poloxamers include PLURONIC® emulsifiers such as PLURONIC® F68, F77, F87, F98, F108, F127, P103, P104, P105, and P123.

Alkoxylated emulsifiers include polyoxyethylenated C8-C30 fatty acid esters (preferably C12-C18) of sorbitan, polyethoxylated C8-C30 (preferably C12-18) fatty alcohols, polyglycerolated C8-C30 (preferably C12-C18) fatty acid esters, polyoxyethylenated compounds having preferably from 2 to 30 moles of ethylene oxide, polyglycerolated compounds having preferably from 2 to 16 moles of glycerol; and combinations thereof. The polyoxyethylenated C8-C30 fatty alcohols may be chosen from C12-C18 fatty alcohols, in particular polyoxyethylenated lauryl alcohol, cetyl alcohol, myristyl alcohol, and stearyl alcohol having from 2 to 30 mol of ethylene oxide, such as: cetyl alcohol polyoxyethylenated with 2 EO (Ceteth-2) (HLB 5.3) cetyl alcohol polyoxyethylenated with 6 EO (Ceteth-6) (HLB 11.1) cetyl alcohol polyoxyethylenated with 10 EO (Ceteth-10) (HLB 12.9) cetyl alcohol polyoxyethylenated with 20 EO (Ceteth-20) (HLB 15.7) cetyl alcohol polyoxyethylenated with 24 EO (Ceteth-24) (HLB 16.3) lauryl alcohol polyoxyethylenated with 2 EO (laureth-2) (HLB 6.1) lauryl alcohol polyoxyethylenated with 3 EO (laureth-3) (HLB 8) lauryl alcohol polyoxyethylenated with 4 EO (laureth-4) (HLB 9.4) lauryl alcohol polyoxyethylenated with 7 EO (laureth-7) (HLB 12.3) lauryl alcohol polyoxyethylenated with 9 EO (laureth-9) (HLB 13.6) lauryl alcohol polyoxyethylenated with 10 EO (laureth-10) (HLB 13.9) lauryl alcohol polyoxyethylenated with 12 EO (laureth-12) (HLB 14.6) lauryl alcohol polyoxyethylenated with 21 EO (laureth-21) (HLB 15.5) lauryl alcohol polyoxyethylenated with 23 EO (laureth-23) (HLB 16.3) stearyl alcohol polyoxyethylenated with 2 EO (Steareth-2) (HLB 4.9) stearyl alcohol polyoxyethylenated with 10 EO (Steareth-10) (HLB 12.4) stearyl alcohol polyoxyethylenated with 20 EO (Steareth-20) (HLB 15.2) stearyl alcohol polyoxyethylenated with 21 EO (Steareth-21) (HLB 15.5).

The polyoxyethylenated C8-C30 fatty acid esters (preferably C12-C18) of sorbitan may be chosen from polyoxyethylenated esters of C12-C18 fatty acids, in particular lauric, myristic, cetylic or stearic acids, of sorbitan especially containing from 2 to 30 mol of ethylene oxide, such as: polyoxyethylenated sorbitan monolaurate (4 EO) (Polysorbate-21) (HLB 13.3) polyoxyethylenated sorbitan monolaurate (20 EO) (Polysorbate-20) (HLB 16.7) polyoxyethylenated sorbitan monopalmitate (20 EO) (Polysorbate-40) (HLB 15.6) polyoxyethylenated sorbitan monostearate (20 EO) (Polysorbate-60) (HLB 14.9) polyoxyethylenated sorbitan monostearate (4 EO) (Polysorbate-61) (HLB 9.6) polyoxyethylenated sorbitan monooleate (20 EO) (Polysorbate-80) (HLB 15). In a preferred embodiment, the cosmetic composition includes one or more nonionic emulsifiers chosen from polyoxyethylenated C8-C30 fatty acid esters (preferably C12-C18) of sorbitan, preferably polyoxyethylenated esters of C12-C18 fatty acids.

The polyglycerolated C8-C30 fatty acid esters may be chosen from polyglycerolated esters of C12-C18 fatty acids, in particular lauric, myristic, palmitic, stearic or isostearic acid, having from 2 to 16 mol of glycerol, such as: polyglyceryl-2 laurate, polyglyceryl-3 laurate, polyglyceryl-4 laurate, polyglyceryl-5 laurate, polyglyceryl-6 laurate, polyglyceryl-10 laurate; polyglyceryl-2 myristate, polyglyceryl-3 myristate, polyglyceryl-4 myristate, polyglyceryl-5 myristate, polyglyceryl-6 myristate, polyglyceryl-10 myristate; polyglyceryl-2 palmitate, polyglyceryl-3 palmitate, polyglyceryl-6 palmitate, polyglyceryl-10 palmitate; polyglyceryl-2 isostearate, polyglyceryl-3 isostearate, polyglyceryl-4 isostearate, polyglyceryl-5 isostearate, polyglyceryl-6 isostearate, polyglyceryl-10 isostearate; polyglyceryl-2 stearate, polyglyceryl-3 stearate, polyglyceryl-4 stearate, polyglyceryl-5 stearate, polyglyceryl-6 stearate, polyglyceryl-8 stearate, polyglyceryl-10 stearate, and combinations thereof.

In a preferred embodiment, the polymeric emulsifier is based on an acrylamido methylpropanesulfonic acid (AMPS) and at least one ethylenically unsaturated monomer comprising at least one hydrophobic part containing from 8 to 30 carbon atoms, in particular from 10 to 30 carbon atoms and more particular from 12 to 22 carbon atoms, in addition, the hydrophobic radical may also comprise at least one alkylene oxide unit and in particular a polyoxyalkylene chain. The polyoxyalkylene chain may be formed from ethylene oxide units and/or propylene oxide units and even more particular be formed solely from ethylene oxide units, preferably, the at least one ethylenically unsaturated monomer is acrylic or methacrylic ester of ethoxylated alcohols RO—(CH2CH2O)mH, in which R is an alkyl radical having 12 to 30 carbon atoms and m is a number from 3 to 35, and CH2=CH—COO—(CH2CH2-COO)nX in which n is a number from 0 to 10 and X is a counterion and is preferably H+, Na+ and/or NH4+. In the case of the copolymers, preferably one or more structural units based on the following comonomers are present in the copolymers: acrylic acid, methacrylic acid, acrylamide, dimethyl-acrylamide, vinylpyrrolidone (VP), hydroxyethyl acrylate, and hydroxyethyl methacrylate. More particularly, the suitable polymeric emulsifier can be copolymer of AMPS and (meth)acrylic ester of ethoxylated C8-C30, or C10-C30, or C12-C22, or C12-C18 fatty alcohol. In some embodiments, the suitable associative polymer can be copolymer of AMPS and (meth)acrylic ester of ethoxylated C8-C30, or C12-C30, or C12-C22, or C12-C18 fatty alcohol, wherein the ethoxylated fatty alcohol in each case comprise 5 to 35, in particular 7 to 30, more in particular 7 to 25 ethylene oxide (EO) radicals. Suitable AMPS copolymers are, for example, ammonium acryloyldimethyltaurate/beheneth-25-methacrylate copolymer such as Aristoflex® HMB from Clariant, ammonium acryloyldimethyltaurate/beheneth-25 methacrylate crosspolymer such as Aristoflex® BLV from Clariant, ammonium Acryloyldimethyltaurate/Steareth-25 Methacrylate crosspolymer such as Aristoflex HMS® by Clariant, ammonium acryloyldimethyltaurate/steareth-8 methacrylate copolymer such as Aristoflex® SNC, Ammonium acryloyldimethyltaurate/laureth-7 methacrylate copolymer such as Aristoflex® LNC.

In a particularly preferred embodiment at least one polymeric emulsifiers is selected from polyacrylamide and acryloyldimethyltaurate polymers such as acrylamide/sodium acryloyldimethyltaurate copolymer (e.g. Sepineo® P600), acrylamide/sodium acryloyldimethyltaurate/acrylic acid copolymer, ammonium acryloyldimethyltaurate/Beheneth-25 methacrylate crosspolymer, ammonium acryloyldimethyltaurate/carboxyethyl acrylate crosspolymer, ammonium acryloyldimethyltaurate/Laureth-7 methacrylate copolymer, ammonium acryloyldimethyltaurate/Steareth-25 methacrylate crosspolymer, ammonium acryloyldimethyltaurate/Steareth-8 methacrylate copolymer, ammonium acryloyldimethyltaurate/vinyl formamide copolymer, ammonium acryloyldimethyltaurate/VP copolymer, ammonium polyacryloyldimethyl taurate, dimethylacrylamide/sodium acryloyldimethyltaurate crosspolymer, HEA/sodium scryloyldimethyltaurate/Steareth-20 methacrylate copolymer, hydroxyethyl acrylate/sodium acryloyldimethyl taurate copolymer, sodium acrylate/acryloyldimethyltaurate/dimethylacrylamide crosspolymer, sodium acrylate/sodium acryloyldimethyl taurate copolymer, sodium acrylate/sodium acryloyldimethyl taurate/acrylamide copolymer, sodium acryloyl dimethyl taurate/PEG-8 diacrylate crosspolymer, sodium acryloyldimethyl taurate/acrylamide/VP copolymer, sodium acryloyl dimethyltaurate/methacrylamidolauric acid copolymer, sodium acryloyldimethyltaurate/VP crosspolymer, sodium polyacryloyldimethyl taurate, or combinations thereof. Particularly preferred polymeric emulsifiers include polyacrylamide, acrylamide/sodium acryloyldimethyltaurate copolymer, or a combination thereof.

Nonlimiting examples of nonionic emulsifiers include poloxamer emulsifiers; polysorbates; alkyl polyglucosides; alcohols, alpha-diols, alkylphenols and esters of fatty acids, being ethoxylated, propoxylated or glycerolated (polyglyceryl-2 isostearate); ethoxylated fatty esters; glyceryl esters of fatty acids; fatty alcohol ethoxylates; alkyl phenol ethoxylates; fatty acid alkoxylates; and combinations thereof.

In various embodiments, the nonionic emulsifiers are selected from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, alkyl and polyalkyl glycosides or polyglycosides, in particular alkyl and polyalkyl glucosides or polyglucosides, alkyl and polyalkyl esters of sucrose, optionally polyoxyethylenated alkyl and polyalkyl esters of glycerol, and optionally polyoxyethylenated alkyl and polyalkyl ethers of glycerol, and combinations thereof. Preferably, the nonionic emulsifiers may be chosen from alkyl and polyalkyl esters of poly(ethylene oxide), alkyl and polyalkyl ethers of poly(ethylene oxide), optionally polyoxyethylenated alkyl and polyalkyl esters of sorbitan, optionally polyoxyethylenated alkyl and polyalkyl ethers of sorbitan, optionally polyoxyethylenated alkyl and polyalkyl esters of glycerol, and optionally polyoxyethylenated alkyl and polyalkyl ethers of glycerol, and combinations thereof.

Nonlimiting examples of sorbitan fatty acid esters include sorbitan isostearate and sorbitan oleate.

In a preferred embodiment, at least one of the one or more nonionic emulsifiers is a sorbitan emulsifier, for example, sorbitan caprylate, sorbitan cocoate, sorbitan diisostearate, sorbitan dioleate, sorbitan distearate, sorbitan isostearate, sorbitan laurate, sorbitan oleate, sorbitan olivate, sorbitan palmitate, sorbitan palmate, sorbitan sesquicaprylate, sorbitan sesquiisostearate, sorbitan sesquioleate, sorbitan sesquistearate, sorbitan stearate, PEG-20 sorbitan cocoate, PEG-40 sorbitan diisostearate, PEG-2 sorbitan isostearate, PEG-5 sorbitan isostearate, PEG-20 sorbitan isostearate, PEG-40 sorbitan laurate, PEG-10 sorbitan laurate, PEG-44 sorbitan laurate, PEG-75 sorbitan laurate, PEG-80 sorbitan laurate, PEG-3 sorbitan oleate, PEG-6 sorbitan oleate, PEG-20 sorbitan oleate, PEG-40 sorbitan oleate, PEG-80 sorbitan palmitate, PEG-3 sorbitan stearate, PEG-4 sorbitan stearate, PEG-6 sorbitan stearate, PEG-40 sorbitan stearate, PEG-60 sorbitan stearate, Polysorbate 20, Polysorbate 21, Polysorbate 40, Polysorbate 60, Polysorbate 61, Polysorbate 65, Polysorbate 80, Polysorbate 81, and Polysorbate 85.

Nonlimiting examples of alkyl and polyalkyl esters of poly(ethylene oxide) include those containing at least one C8-C30 alkyl radical, with a number of ethylene oxide (EO) units ranging from 2 to 200. Mention may be made, for example, of (INCI name) PEG-20 stearate, PEG-40 stearate, PEG-100 stearate, PEG-20 laurate, PEG-8 laurate, PEG-40 laurate, PEG-150 distearate, PEG-7 cocoate, PEG-9 cococate, PEG-8 oleate, PEG-10 oleate and PEG-40 hydrogenated castor oil.

Nonlimiting examples of alkyl and polyalkyl ethers of poly(ethylene oxide) include those containing at least one C8-C30 alkyl radical, with a number of ethylene oxide (EO) units ranging from 3 to 200. Mention may be made, for example, of laureth-3, laureth-4, laureth-7, laureth-23, ceteth-5, ceteth-7, ceteth-15, ceteth-23, oleth-5, oleth-7, oleth-10, oleth-12, oleth-20, oleth-50, phytosterol 30 EO, steareth-6, steareth-20, steareth-21, steareth-40, steareth-100, beheneth 100, ceteareth-7, ceteareth-10, ceteareth-15, ceteareth-25, pareth-3, pareth-23, C12-15 pareth-3, C12-13 pareth-4, C12-13 pareth-23, trideceth-3, trideceth-4, trideceth-5, trideceth-6, trideceth-7 and trideceth-10, and combinations thereof.

Nonlimiting examples of polyoxyethylenated alkyl and polyalkyl esters of sorbitan include those with a number of ethylene oxide (EO) units ranging from 0 to 100. Mention may be made, for example, of sorbitan laurate, sorbitan laurate 4 EO, sorbitan laurate 20 EO (polysorbate 20), sorbitan palmitate 20 EO (polysorbate 40), sorbitan stearate 20 EO (polysorbate 60), sorbitan oleate 20 EO (polysorbate 80) and sorbitan trioleate 20 EO (polysorbate 85). Polyoxyethylenated alkyl and polyalkyl ethers of sorbitan that are preferably used are those with a number of ethylene oxide (EO) units ranging from 0 to 100.

The cosmetic composition may include one or more alkanolamides. Nonlimiting examples alkanolamides include fatty acid alkanolamides. The fatty acid alkanolamides may be fatty acid monoalkanolamides or fatty acid dialkanolamides or fatty acid isoalkanolamides, and may have a C2-8 hydroxyalkyl group (the C2-8 chain can be substituted with one or more than one —OH group). Non-limiting examples include fatty acid diethanolamides (DEA) or fatty acid monoethanolamides (MEA), fatty acid monoisopropanolamides (MIPA), fatty acid diisopropanolamides (DIPA), and fatty acid glucamides (acyl glucamides). Nonlimiting examples of fatty alkanolamides include those formed by reacting an alkanolamine and a C6-C36 fatty acid, for example: oleic acid diethanolamide, myristic acid monoethanolamide, soya fatty acids diethanolamide, stearic acid ethanolamide, oleic acid monoisopropanolamide, linoleic acid diethanolamide, stearic acid monoethanolamide (Stearamide MEA), behenic acid monoethanolamide, isostearic acid monoisopropanolamide (isostearamide MIPA), erucic acid diethanolamide, ricinoleic acid monoethanolamide, coconut fatty acid monoisopropanolamide (cocoamide MIPA), coconut acid monoethanolamide (Cocamide MEA), palm kernel fatty acid diethanolamide, coconut fatty acid diethanolamide, lauric diethanolamide, polyoxyethylene coconut fatty acid monoethanolamide, coconut fatty acid monoethanolamide, lauric monoethanolamide, lauric acid monoisopropanolamide (lauramide MIPA), myristic acid monoisopropanolamide (Myristamide MIPA), coconut fatty acid diisopropanolamide (cocamide DIPA), and combinations thereof. In some instances, the fatty acid alkanolamides may preferably be selected from cocamide MIPA, cocamide DEA, cocamide MEA, cocamide DIPA, and combinations thereof.

The one or more nonionic emulsifiers may include one or more acyl glucamides, for example, acyl glucamides having a carbon chain length of 8 to 20. Nonlimiting examples include lauroyl/myristoyl methyl glucamide, capryloyl/capryl methyl glucamide, lauroyl methyl glucamide, myristoyl methyl glucamide, capryloyl methyl glucamide, capryl methyl glucamide, cocoyl methyl glucamide, capryloyl/caproyl methyl glucamide, cocoyl methyl glucamide, lauryl methylglucamide, oleoyl methylglucamide oleate, stearoyl methylglucamide stearate, sunfloweroyl methylglucamide, and tocopheryl succinate methylglucamide.

The cosmetic composition may include one or more alkyl polyglucosides. Non-limiting examples of alkyl polyglucosides include those having the following formula:

R1-O—(R2O)n-Z(x)
wherein R1 is an alkyl group having 8-18 carbon atoms;
R2 is an ethylene or propylene group;
Z is a saccharide group with 5 to 6 carbon atoms;
n is an integer from 0 to 10; and
x is an integer from 1 to 5.

Preferred alkyl polyglucosides include lauryl glucoside, octyl glucoside, decyl glucoside, coco glucoside, caprylyl/capryl glucoside, and sodium lauryl glucose carboxylate. Typically, the at least one alkyl poly glucoside compound is selected from the group consisting of lauryl glucoside, decyl glucoside coco glucoside, or combinations thereof.

In various embodiments, the nonionic emulsifiers may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C8-C24, preferably C12-C22, fatty acid or acids and alkoxylated derivatives thereof, preferably with a number of alkyleneoxide of from 10 to 200, and more preferably from 10 to 100; polyethylene glycol esters of a C8-C24, preferably C12-C22, fatty acid or acids and alkoxylated derivatives thereof, preferably with a number of alkyleneoxide of from 10 to 200, and more preferably from 10 to 100; sorbitol esters of a C8-C24, preferably C12-C22, fatty acid or acids and alkoxylated derivatives thereof, preferably with a number of alkyleneoxide of from 10 to 200, and more preferably from 10 to 100; sugar (sucrose, glucose, alkylglycose) esters of a C8-C24, preferably C12-C22, fatty acid or acids and alkoxylated derivatives thereof, preferably with a number of alkyleneoxide of from 10 to 200, and more preferably from 10 to 100; ethers of fatty alcohols; ethers of sugar and a C8-C24, preferably C12-C22, fatty alcohol or alcohols; and combinations thereof.

Examples of ethoxylated fatty esters that may be mentioned include the adducts of ethylene oxide with esters of lauric acid, palmitic acid, stearic acid or behenic acid, and combinations thereof, especially those containing from 9 to 100 oxyethylene groups, such as PEG-9 to PEG-50 laurate (as the CTFA names: PEG-9 laurate to PEG-50 laurate); PEG-9 to PEG-50 palmitate (as the CTFA names: PEG-9 palmitate to PEG-50 palmitate); PEG-9 to PEG-50 stearate (as the CTFA names: PEG-9 stearate to PEG-50 stearate); PEG-9 to PEG-50 palmitostearate; PEG-9 to PEG-50 behenate (as the CTFA names: PEG-9 behenate to PEG-50 behenate); polyethylene glycol 100 EO monostearate (CTFA name: PEG-100 stearate); and combinations thereof.

In a preferred embodiment, the cosmetic composition includes one or more glyceryl esters of fatty acids, for example a glyceryl stearate (glyceryl mono-, di- and/or tristearate) (CTFA name: glyceryl stearate) or glyceryl ricinoleate and combinations thereof can in particular be cited. As glyceryl esters of C8-C24 alkoxylated fatty acids, polyethoxylated glyceryl stearate (glyceryl mono-, di- and/or tristearate) such as PEG-20 glyceryl stearate and PEG-30 glyceryl stearate can for example be cited. Mixtures of these surfactants, such as for example the product containing glyceryl stearate and PEG-100 stearate, marketed under the name ARLACEL 165 by Uniqema, and the product containing glyceryl stearate (glyceryl mono- and distearate) and potassium stearate marketed under the name TEG1N by Goldschmidt (CTFA name: glyceryl stearate SE), can also be used.

Suitable alkoxylated monoacid emulsifiers include, but are not limited to: butoxynol-5 carboxylic acid, butoxynol-19 carboxylic acid, capryleth-4 carboxylic acid, capryleth-6 carboxylic Acid, capryleth-9 carboxylic acid, ceteareth-25 carboxylic acid, coceth-7 carboxylic acid, C9-11 pareth-6 carboxylic acid, C11-15 pareth-7 carboxylic acid, C12-13 pareth-5 carboxylic acid, C12-13 pareth-8 carboxylic acid, C12-13 pareth-12 carboxylic acid, C12-15 pareth-7 carboxylic acid, C12-15 pareth-8 carboxylic acid, C14-15 pareth-8 carboxylic acid, deceth-7 carboxylic acid, laureth-3 carboxylic acid, laureth-4 carboxylic Acid, laureth-5 carboxylic acid, laureth-6 carboxylic acid, laureth-8 carboxylic acid, laureth-10 carboxylic acid, laureth-11 carboxylic acid, laureth-12 carboxylic acid, laureth-13 carboxylic acid, laureth-14 carboxylic acid, laureth-17 carboxylic acid, PPG-6-laureth-6 carboxylic acid, PPG-8-steareth-7 carboxylic acid, myreth-3 carboxylic acid, myreth-5 carboxylic acid, Nonoxynol-5 carboxylic acid, nonoxynol-8 carboxylic acid, Nonoxynol-10 carboxylic acid, octeth-3 carboxylic acid, octoxynol-20 carboxylic acid, oleth-3 carboxylic acid, oleth-6 carboxylic acid, oleth-10 carboxylic acid, PPG-3-deceth-2 carboxylic acid, capryleth-2 carboxylic acid, Ceteth-13 carboxylic acid, deceth-2 carboxylic acid, hexeth-4 carboxylic acid, isosteareth-6 carboxylic acid, isosteareth-11 carboxylic acid, trudeceth-3 carboxylic acid, trideceth-6 carboxylic acid, trideceth-8 carboxylic acid, trideceth-12 carboxylic acid, trideceth-3 carboxylic acid, trideceth-4 carboxylic acid, trideceth-7 carboxylic acid, trideceth-15 carboxylic acid, trideceth-19 carboxylic acid, undeceth-5 carboxylic acid and combinations thereof.

The total amount of the one or more emulsifiers in the cosmetic composition will vary but is typically about 0.1 to about 10 wt. %, based on the total weight of the cosmetic composition. In further embodiments, the cosmetic composition includes about 0.1 to about 8 wt. %, about 0.1 to about 5 wt. %, about 0.1 to about 3 wt. %, about 0.5 to about 10 wt. %, about 0.5 to about 8 wt. %, about 0.5 to about 5 wt. %, about 0.5 to about 3 wt. %, about 1 to about 10 wt. %, about 1 to about 8 wt. %, about 1 to about 5 wt. %, about 1 to about 4 wt. %, about 2 to about 10 wt. %, about 2 to about 8 wt. %, or about 2 to about 5 wt. % of one or more emulsifiers, based on the total weight of the cosmetic composition. Preferably, the cosmetic composition includes about 0.5 to about 10 wt. %, more preferably about 1 to about 8 wt. %, and even more preferably, about 2 to about 6 wt. % of one or more emulsifiers, based on the total weight of the cosmetic composition.

### Water

In accordance with the disclosure, the cosmetic composition is a water-based composition.

In accordance with the various embodiments, water is present in the composition at a concentration, by weight, based on the total weight of the composition, of about 40% or greater, alternatively about 50% or greater, alternatively about 98% or less, alternatively from about 40% to about 98%, alternatively from about 45% to about 96%, alternatively from about 50% to about 94%, alternatively from about 55% to about 90%, alternatively from about 60% to about 88%, or any suitable value, range, or sub-range thereof. Thus, the water may be present in an amount from about 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97% to about 98%.

### Water Soluble Solvent

The term “water soluble solvent” is interchangeable with the term “water soluble organic solvent,” and means an organic solvent that is water soluble or can be solubilized in water and is a liquid at 25° C. and at atmospheric pressure (760 mmHg). For example, water soluble solvents may have a solubility of at least 50% in water under these conditions. Preferably, however, the water soluble solvent have a solubility of at least 60%, 70%, 80%, or 90%.

Nonlimiting examples of water soluble solvents include glycerin, C₂-C₆ mono-alcohols, polyols, glycols, glycol ethers (for example, monomethyl, monoethyl, and monobutyl ethers of ethylene glycol or propylene glycol), propylene glycol, butylene glycol, hexylene glycol, caprylyl glycol, dipropylene glycol, alkyl ethers of diethylene glycol such as monoethyl ether or monobutyl ether of diethylene glycol (e.g., diethylene glycol monobutyl ether and diethylene glycol monoethyl ether), dipropylene glycol, and alkyl ethers of diethylene glycol, for example monoethyl ether or monobutyl ether of diethylene glycol.

In various embodiment, one or more water soluble solvents are selected from alkanediols (polyhydric alcohols or polyols) such as glycerin, 1,2,6-hexanetriol, trimethylolpropane, ethylene glycol, propylene glycol, diethylene glycol, triethylene glycol, caprylyl glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or combinations thereof.

Nonlimiting examples of polyols (polyhydric alcohols) include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a combination thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, of combinations thereof.

The total amount of the one or more water soluble solvents in the cosmetic composition will vary but is typically about 1 to about 30 wt. %, based on the total weight of the cosmetic composition. For example, the cosmetic composition may include water-soluble solvents in an amount of about 1 to about 30 wt. %, about 1 to about 25 wt. %, about 1 to about 20 wt. %, about 1 to about 18 wt. %, about 1 to about 16 wt. %, about 1 to about 14 wt. %, about 1 to about 12 wt. %, about 1 to about 10 wt. %; about 5 to about 30 wt. %, about 5 to about 25 wt. %, about 5 to about 20 wt. %, about 5 to about 18 wt. %, about 5 to about 16 wt. %, about 5 to about 14 wt. %, about 5 to about 12 wt. %, about 5 to about 10 wt. %; about 10 to about 30 wt. %, about 10 to about 25 wt. %, about 10 to about 20 wt. %, about 10 to about 18 wt. %, about 10 to about 16 wt. %, about 10 to about 14 wt. %; about 12 to about 30 wt. %, about 12 to about 25 wt. %, about 12 to about 20 wt. %, about 12 to about 18 wt. %, about 12 to about 16 wt. %; about 14 to about 30 wt. %, about 14 to about 25 wt. %, about 14 to about 20 wt. %, about 14 to about 18 wt. %; about 16 to about 30 wt. %, about 16 to about 25 wt. %, about 16 to about 20 wt. %; about 18 to about 30 wt. %, about 18 to about 25 wt. %, about 18 to about 20 wt. %, including ranges and subranges thereof, based on the total weight of the cosmetic composition.

### Skin Active Agents

The cosmetic compositions may, optionally, include one or more skin active agents, such as anti-aging agent, anti-wrinkle actives, anti-oxidants, humectants, moisturizing ingredients, depigmenting agents, and/or agents for treating oily skin etc. The skin active agents may be included in the cosmetic composition in an amount ranging from greater than zero to about to about 10 wt. %, based on the total weight of the composition. For example, the total amount of skin active agents may be from greater than zero to about 9 wt. %, greater than zero to about 8 wt. %, greater than zero to about 7 wt. %, greater than zero to about 6 wt. %, greater than zero to about 5 wt. %, greater than zero to about 4 wt. %, greater than zero to about 3 wt. %, greater than zero to about 2 wt. %; from about 10 ppm to about 10 wt. % (100,000 ppm), about 10 ppm to about 5 wt. % (50,000 ppm), about 10 ppm to about 2.5 wt. % (25,000 ppm), about 10 ppm to about 1 wt. % (10,000 ppm), about 10 ppm to about 0.5 wt. % (5,000 ppm), about 10 ppm to about 0.3 wt. % (3,000 ppm), about 10 ppm to about 0.2 wt. % (2,000 ppm), about 10 ppm to about 0.1 wt. % (1,000 ppm), about 10 ppm to 500 ppm; about 0.05 to about 10 wt. %, about 0.05 to about 5 wt. %, about 0.05 to about 2.5 wt. %, about 0.05 to about 1 wt. %, about 0.05 to about 0.5 wt. %; about 0.1 to about 10 wt. %, about 0.1 to about 5 wt. %, about 0.1 to about 2.5 wt. %, about 0.1 to about 1 wt. %, about 0.1 to about 0.5 wt. %; from about 1 to about 10 wt. %, about 1 to about 8 wt. %, about 1 to about 6 wt. %, about 1 to about 5 wt. %, about 1 to about 4 wt. %, about 1 to about 3 wt. %; from about 2 to about 10 wt. %, about 2 to about 8 wt. %, about 2 to about 6 wt. %, about 2 to about 5 wt. %, about 2 to about 4 wt. %; from about 3 to about 10 wt. %, about 3 to about 8 wt. %, about 3 to about 6 wt. %, about 3 to about 5 wt. %; from about 4 to about 10 wt. %, about 4 to about 8 wt. %, about 4 to about 6 wt. %, including ranges and subranges therebetween, based on the total weight of the cosmetic composition.

A non-limiting discussion of skin active agents that may, in some cases, be included in the cosmetic composition is provided below:

Humectants and/or Moisturizing Ingredients Others than Hydroxyethyl Urea

Examples of humectants and/or moisturizing ingredients include glycerol and its derivatives, lactic acid, hyaluronic acid, AHA, BHA, sodium pidolate, xylitol, serine, sodium lactate, ectoin and its derivatives, chitosan and its derivatives, collagen, plankton, an extract of Imperata cylindra sold under the name Moist 24 by Sederma, homopolymers of acrylic acid as Lipidure-HM of NOF Corporation, beta-glucan and in particular sodium carboxymethyl beta-glucan Mibelle-AG-Biochemistry, a mixture of oils passionflower, apricot, corn, and rice bran sold by Nestle under the name NutraLipids, a C-glycoside derivatives, in particular the C-13-D-xylopyranoside-2-hydroxypropane in the form of a solution at 30% by weight of active material in a water/propylene glycol mixture (60/40 wt %) as the product produced by the company Chimex under the trade name “Mexoryl SBB”, a rose hip oil marketed by Nestle, a micro-algae extract Prophyridium cruentum enriched with zinc, marketed under the name by Vincience Algualane Zinc spheres of collagen and chondroitin sulfate of marine origin (Atelocollagen) sold by the company Engelhard Lyon under the name Marine Filling Spheres, hyaluronic acid spheres such as those marketed by Engelhard Lyon, and arginine.

### Depigmenting Agents

Depigmenting agents that may be incorporated in the cosmetic composition include those chosen from alpha and beta arbutin, ferulic acid, lucinol and its derivatives, kojic acid, resorcinol and derivatives thereof, tranexamic acid and derivatives thereof, gentisic acid, homogentisic, methyl gentisate or homogentisate, dioic acid, D pantheteine calcium sulphonate, lipoic acid, ellagic acid, vitamin B3, linoleic acid and its derivatives, certain compounds derived from plants such as chamomile, bearberry, the aloe family (vera, ferox, bardensis), mulberry, skullcap, a water kiwi fruit (Actinidia chinensis) marketed by Gattefosse, an extract of Paeonia suffruticosa root, such as that sold by Ichimaru Pharcos under the name Liquid Botanpi Be an extract of brown sugar (Saccharum officinarum) such as molasses extract marketed by Taiyo Kagaku under the name Liquid Molasses, without this list being exhaustive. Particular depigmenting agents include alpha and beta arbutin, ferulic acid, kojic acid, resorcinol and derivatives, D pantheteine calcium sulfonate, lipoic acid, ellagic acid, vitamin B3, a water kiwi fruit (Actinidia chinensis) marketed by Gattefosse, an extract of Paeonia suffruticosa root, such as that sold by the company Ichimaru Pharcos under the name Botanpi Liquid B.

### Anti-Wrinkle Active

The cosmetic composition may include one or more anti-wrinkle actives. The term “anti-wrinkle active” refers to a natural or synthetic compound producing a biological effect, such as the increased synthesis and/or activity of certain enzymes, when brought into contact with an area of wrinkled skin, this has the effect of reducing the appearance of wrinkles and/or fine lines. Exemplary anti-wrinkle actives may be chosen from: desquamating agents, anti-glycation agents, inhibitors of NO-synthase, agents stimulating the synthesis of dermal or epidermal macromolecules and/or preventing their degradation, agents for stimulating the proliferation of fibroblasts and/or keratinocytes, or for stimulating keratinocyte differentiation reducing agents; muscle relaxants and/or dermo-decontracting agents, anti-free radical agents, and mixtures thereof. Examples of such compounds are: adenosine and its derivatives and retinoids (such as, retinol palmitate and retinol), ascorbic acid and its derivatives such as magnesium ascorbyl phosphate and ascorbyl glucoside; nicotinic acid and its precursors such as nicotinamide; ubiquinone; glutathione and precursors thereof such as L-2-oxothiazolidine-4-carboxylic acid, the compounds C-glycosides and their derivatives as described in particular in EP-1345919, in particular C-beta-D-xylopyranoside-2-hydroxy-propane as described in particular in EP-1345919, plant extracts including sea fennel and extracts of olive leaves, as well as plant and hydrolysates thereof such as rice protein hydrolysates or soybean proteins; algal extracts and in particular laminaria, bacterial extracts, the sapogenins such as diosgenin and extracts of Dioscorea plants, in particular wild yam, comprising: the a-hydroxy acids, f3-hydroxy acids, such as salicylic acid and n-octanoyl-5-salicylic oligopeptides and pseudodipeptides and acyl derivatives thereof, in particular acid {2-[acetyl-(3-trifluoromethyl-phenyl)-amino]-3-methyl-}acetic acid and lipopeptides marketed by the company under the trade names SEDERMA Matrixyl 500 and Matrixyl 3000; lycopene, manganese salts and magnesium salts, especially gluconates, and mixtures thereof. In at least one case, the cosmetic composition includes adenosine derivatives, such as non-phosphate derivatives of adenosine, such as in particular the 2′-deoxyadenosine, 2′,3′-adenosine isopropoylidene; the toyocamycine, 1-methyladenosine, N-6-methyladenosine; adenosine N-oxide, 6-methylmercaptopurine riboside, and the 6-chloropurine riboside. Other derivatives include adenosine receptor agonists such as adenosine phenylisopropyl (“PIA”), 1-methylisoguanosine, N6-cyclohexyladenosine (CHA), N6-cyclopentyladenosine (CPA), 2-chloro-N6-cyclopentyladenosine, 2-chloroadenosine, N6-phenyladenosine, 2-phenylaminoadenosine, MECA, N 6-phenethyladenosine, 2-p-(2-carboxy-ethyl) phenethyl-amino-5′-N-ethylcarboxamido adenosine (CGS-21680), N-ethylcarboxamido-adenosine (NECA), the 5′(N-cyclopropyl)-carboxamidoadenosine, DPMA (PD 129.944) and metrifudil.

### Skin Active Agent for Oily Skin

The cosmetic composition may, optionally, include a skin active agent that addresses oily skin. These agents can be sebo-regulating or antiseborrhoeic agents capable of regulating the activity of sebaceous glands. Exemplary skin active agents for addressing oily skin include: retinoic acid, retinol, benzoyl peroxide, sulfur, vitamin B6 (pyridoxine or) chloride, selenium, samphire—the cinnamon extract blends, tea and octanoylglycine such as—15 Sepicontrol A5 TEA from Seppic—the mixture of cinnamon, sarcosine and octanoylglycine marketed especially by Seppic under the trade name Sepicontrol A5—zinc salts such as zinc gluconate, zinc pyrrolidonecarboxylate (or zinc pidolate), zinc lactate, zinc aspartate, zinc carboxylate, zinc salicylate 20, zinc cysteate; —derivatives particularly copper and copper pidolate as Cuivridone Solabia—extracts from plants of Arnica montana, Cinchona succirubra, Eugenia caryophyllata, Humulus lupulus, Hypericum perforatum, Mentha pipenta 25 Rosmarinus officinalis, Salvia officinalis and Thymus vulgaris, all marketed for example by Maruzen—extracts of meadowsweet (Spiraea ulmaria), such as that sold under the name Sebonormine by Silab—extracts of the alga Laminaria saccharina, such as that sold under the 30 name Phlorogine by Biotechmarine—the root extracts of burnet mixtures (Sanguisorba officinalis/Poterium officinale), rhizomes of ginger (Zingiber officinalis) and cinnamon bark (Cinnamomum cassia), such as that sold under the name Sebustop by Solabia—extracts of flaxseed such as that sold under the name Linumine by Lucas Meyer—Phellodendron extracts such as those sold under the name Phellodendron extract BG by Maruzen or Oubaku liquid B by Ichimaru Pharcos—of argan oil mixtures extract of Serenoa serrulata (saw palmetto) extract and sesame seeds such as that sold under the name Regu SEB by Pentapharm—mixtures of extracts of willowherb, of Terminalia chebula, nasturtium and of bioavailable zinc (microalgae), such as that sold under the name Seborilys Green Tech; —extracts of Pygeum afrianum such as that sold under the name Pygeum afrianum sterolic lipid extract by Euromed—extracts of Serenoa serrulata such as those sold under the name Viapure Sabal by Actives International, and those sold by the company Euromed—of extracts of plantain blends, Berberis aquifolium and sodium salicylate 20 such as that sold under the name Seboclear Rahn—extract of clove as that sold under the name Clove extract powder by Maruzen—argan oil such as that sold under the name Lipofructyl Laboratories Serobiologiques; 25—lactic protein filtrates, such as that sold under the name Normaseb by Sederma—the seaweed laminaria extracts, such as that sold under the name Laminarghane by Biotechmarine—oligosaccharides seaweed Laminaria digitata, such as that sold under the name Phycosaccharide 30 AC by the company Codif—extracts of sugar cane such as that sold under the name Policosanol by the company Sabinsa, the sulfonated shale oil, such as that sold under the name Ichtyol Pale by Ichthyol—extracts of meadowsweet (Spiraea ulmaria) such as that sold under the name Cytobiol Ulmaire by societeLibiol—sebacic acid, especially sold in the form of a sodium polyacrylate gel under the name Sebosoft by Sederma—glucomannans extracted from konjac tuber and modified with alkylsulfonate chains such as that sold under the name Biopol Beta by Arch Chemical—extracts of Sophora angustifolia, such as those sold under the name Sophora powder or Sophora extract by Bioland—extracts of cinchona bark succirubra such as that sold under the name Red Bark HS by Alban Muller—extracts of Quillaja saponaria such as that sold under the name 15 Panama wood HS by Alban Muller—glycine grafted onto an undecylenic chain, such as that sold under the name Lipacide UG OR by SEPPIC—the mixture of oleanolic acid and nordihydroguaiaretic acid, such as that sold under the form of a gel under the name AC.Net by Sederma; 20—phthalimidoperoxyhexanoic acid—citrate tri (C12-C13) sold under the name COSMACOL® ECI by Sasol; trialkyl citrate (C14-C15) sold under the name COSMACOL® ECL by Sasol—10-hydroxydecanoic acid, including mixtures acid-hydroxydecanoic October 25, sebacic acid and 1,10-decandiol such as that sold under the name Acnacidol BG by Vincience and mixtures thereof.

### Antioxidants

Vitamin C and derivatives may be used, including ascorbic acid, sodium ascorbate, and the fat soluble esters tetrahexyldecyl ascorbate and ascorbyl palmitate, magnesium ascorbyl phosphate, ascorbyl-glucoside, glucosamine ascorbate, ascorbyl acetate, etc. Additionally, extracts from plants containing a high amount of vitamin C such as camu berry (Myrciaria dubia), acerola, Emblica officinalis, and bioflavonoids from rose hip and citrus may be used including watersoluble bioflavonoids such as hesperidin methyl chalcone may also be used.

Sesame (Sesamum indicum) or sesame lignan may also be added. Sesame and its lignans (the fibrous compounds associated with the sesame) act as antioxidants. Sesame seed lignans significantly enhance vitamin E activity.

Other antioxidants include tocopherols (e.g. d-α-tocopherol, d-β-tocopherol, d-γ-tocopherol, d-delta-tocopherol), tocotrienols (e.g. d-α-tocotrienol, d-β-tocotrienol, d-γ-tocotrienol, d-delta-tocotrienol,) and vitamin E (α-tocopherol acetate). These compounds may be isolated from natural sources, prepared by synthetic means, or mixtures thereof. Tocotrienol-enriched vitamin E preparations may be obtained by fractionating vitamin E preparations to remove a portion of tocopherols and recover a preparation more highly concentrated in tocotrienol. Useful tocotrienols are natural products isolated, for example, from wheat germ oil, grain, or palm oil using high performance liquid chromatography, or isolated by alcohol extraction and/or molecular distillation from barley, brewer's grain or oats. As used herein, the term “tocotrienols” includes tocotrienol-rich-fractions obtained from these natural products as well as the pure compounds. The increased glutathione peroxidase activity protects the skin from oxidative damage.

In addition, carotenoids, particularly the xanthophyll type, are also useful antioxidants that can be used. The xanthopyll type carotenoids include molecules, such as lutein, canthaxantin, cryptoxanthin, zeaxanthin and astaxanthin. Xanthophylls protect compounds, such as vitamin A, vitamin E, and other carotenoids.

### Flavonoids

The active agent may be an antioxidant selected from the group of flavonoids. In some instances, the flavonoid is a flavanone (derivative of 2,3-dihydro-2-phenylchromen-4-one). Flavones include: Butin, Eriodictyol, Hesperetin, Hesperidin, Homoeriodictyol, Isosakuranetin, Naringenin, Naringin, Pinocembrin, Poncirin, Sakuranetin, Sakuranin, and Sterubin. The flavonoid may be a flavanonol (derivative of 3-hydroxy-2,3-dihydro-2-phenylchromen-4-one). Flavanols include: Taxifolin, Aromadedrin, Chrysandroside A, Chrysandroside B, Xeractinol, Astilbin, and Fustin. The flavonoid may be a flavone (derivative of 2-phenylchromen-4-one). Flavones include: Apigenin, Luteolin, Tangeritin, Chrysin, Baicalein, Scutellarein, Wogonin, Synthetic Flavones: Diosmin, and Flavoxate. The flavonoid may be a flavonol (derivative of 3-hydroxy-2-phenylchromen-4-one). Flavonols include: 3-Hydroxyflavone, Azaleatin, Fisetin, Galangin, Gossypetin, Kaempferide, Kaempferol, Isorhamnetin, Morin, Myricetin, Natsudaidain, Pachypodol, Quercetin, Rhamnazin, Rhamnetin, Azalein, Hyperoside, Isoquercitin, Kaempferitrin, Myricitrin, Quercitrin, Robinin, Rutin, Spiraeoside, Xanthorhamnin, Amurensin, Icariin, and Troxerutin. The flavonoid may be a flavan-3-ol (derivatives of 2-phenyl-3,4-dihydro-2H-chromen-3-ol). Flavan-3-ols include: Catechin, Epicatechin, Epigallocatechin, Epicatechin gallate, Epigallocatechin gallate, Epiafzelechin, Fisetinidol, Guibourtinidol, Mesquitol, and Robinetinidol. The flavonoid may be a flavan-4-ol (derivative of 2-phenylchroman-4-ol). Flavan-4-ols include: Apiforol and Luteoforol. The flavonoid may be an isoflavone (derivative of 3-phenylchromen-4-one). Isoflavones include: Genistein, Daidzein, Biochanin A, Formononetin, and the Equol metabolite from Daidzein.

The antioxidant may be an anthocyanidin (derivative of 2-phenylchromenylium cation). Anthocyanidins include: Aurantinidin, Cyanidin, Delphinidin, Europinidin, Luteolinidin, Pelargonidin, Malvidin, Peonidin, Petunidin, Rosinidin, and Xanthone.

The antioxidant may be a Dihydrochalcone (derivative of 1,3-diphenyl-1-propanone). Dihydrochalcones include: Phloretin, Dihydrochalcone phloretin Phlorizin, Aspalathin, Naringin dihydrochalcone, Neohesperidin dihydrochalcone, and Nothofagin. Without limiting the mode of action of the invention, dihydrochalcones may exert an antioxidant effect by reducing reactive free radicals, like reactive oxygen and reactive nitrogen species.

The antioxidant may be an anthocyanin. Anthocyanins and their derivatives are antioxidants. Anthocyanins encompasses a class of flavonoid compounds that are naturally occurring, water-soluble compounds, responsible for the red, purple, and blue colors of many fruits, vegetables, cereal grains, and flowers. Additionally, anthocyanins are collagenase inhibitors. The inhibition of collagenase helps in the prevention and reduction of wrinkles, increase in skin elasticity, etc., which are caused by a reduction in skin collagen. The anthocyanins may be obtained from any portion of various plant sources, such as the fruit, flower, stem, leaves, root, bark, or seeds. One of skill in the art will understand that certain portions of the plant may contain higher natural levels of anthocyanins, and, therefore, those portions are used to obtain the desired anthocyanins. In some instances, antioxidants may include one or more betacyanin. Betacyanins, like anthocyanins, may be obtained from natural sources and are antioxidants.

The antioxidant may be a Phenylpropanoid (derivatives of cinnamic acid). Phenylpropanoids include: Cinnamic acid, Caffeic acid, Ferulic acid, Trans-ferulic acid (including its antioxidant pharmacore 2,6-dihydroxyacetophenome), 5-Hydroxyferulic acid, Sinapic acid, Coumaryl alcohol, Coniferyl alcohol, Sinapyl alcohol, Eugenol, Chavicol, Safrole, P-coumaric acid, and Sinapinic acid. Without limiting the mode of action of the invention, Phenylpropanoids may neutralize free radicals.

The antioxidant may be a Chalcone (derivative of 1,3-diphenyl-2-propen-1-one). Chalcones include: Butein, Okanin, Carthamin, Marein, Sophoradin, Xanthohumol, Flavokvain A, Flavokavain B, Flavokavin C, and synthetic Safalcone.

The antioxidant may be a Curcuminoid. Curcuminoids include: Curcumin, Desmethoxycurcumin, bis-Desmethoxycurcumin, Tetrahydrocurcumin, and Tetrahydrocurcuminoids. Curcumin and tetrahydrocurcuminoids may be derived from rhizomes of Curcuma longa. Tetrahydrocurcumin, a metabolite of curcumin, has been found to be a more potent antioxidant and more stable compared to curcumin.

The antioxidant may be a Tannin. Tannins include: Tannin, Terflavin B, Glucogallin, Dgallic acid, and Quercitannic acid.

The antioxidant may be a stilbenoid. Stilbenoids include: Resveratrol, Pterostilbene, and Piceatannol. Resveratrol may include, but is not limited to, 3,5,4′-trihydroxystilbene, 3,4,3′,5′-tetrahydroxystilbene (piceatannol), 2,3′,4,5′-tetrahydroxystilbene (oxyresveratrol), 4,4′-dihydroxystilbene, and alpha and beta glucoside, galactoside and mannoside derivatives thereof.

The antioxidant may be a Coumarin (derivatives of 2H-chromen-2-one). Coumarins include: 4-Hydroxycoumarin, Umbelliferone, Aesculetin, Herniarin, Auraptene, and Dicoumarol.

The antioxidant may be a Carotenoid. Carotenoids include: beta-Carotene, alpha-Carotene, gamma-Carotene, beta-Cryptoxanthin, Lycopene, Lutein, and Idebenone. Sesame (Sesamum indicum) or sesame lignan may also be added. Sesame and its lignans (the fibrous compounds associated with the sesame) act as antioxidants. Sesame seed lignans significantly enhance vitamin E activity.

The antioxidant may be: a Xanthone, Butylated Hydroxytoluene, 2,6-Di-tert-butylphenol, 2,4-Dimethyl-6-tert-butylphenol, Gallic acid, Eugenol, Uric acid, alpha-Lipoic acid, Ellagic acid, Chicoric acid, Chlorogenic acid, Rosmarinic acid, Salicylic acid, Acetylcysteine, S-Allyl cysteine, Barbigerone, Chebulagic acid, Edaravone, Ethoxyquin, Glutathione, Hydroxytyrosol, Idebenone, Melatonin, N-Acetylserotonin, Nordihydroguaiaretic acid, Oleocanthal, Oleuropein, Paradol, Piceatannol, Probucol, Propyl gallate, Protocatechuic acid, Pyritinol, Rutin, Secoisolariciresinol diglucoside, Sesamin, Sesamol, Silibinin, Silymarin, Theaflavin, Theaflavin digallate, Thmoquinone, Trolox, Tyrosol, Polyunsaturated fatty acids, and sulfur-based antioxidants such as Methionine or Lipoic acid.

### pH Adjuster(s)

The cosmetic composition may include one or more pH adjusters to increase or decrease the overall pH of the cosmetic composition. For example, one or more acids may be included to decrease the pH of the cosmetic composition. Examples of suitable acids for decreasing the pH of the cosmetic composition include, but are not limited to, citric acid, acetic acid, and the like. The cosmetic composition may include one or more bases, such as sodium hydroxide, potassium hydroxide and the like, to decrease the pH of the cosmetic composition. Additional or alternative acids and bases that are suitable for adjusting the pH of the cosmetic composition are readily known to one of ordinary skill in the art.

The cosmetic composition may, desirably, have a pH of about 4.5 to about 7, preferably about 5 to about 6.5 or about 5.5 to about 6.5. In one instance, the pH of the cosmetic composition is 6 or about 6. The amount of the pH adjuster in the cosmetic composition may be based on the desired pH of the final cosmetic composition and/or product. For example, the total amount of the pH adjuster may range from about 0.05 to about 20 wt. %, based on the total weight of the composition. In some instances, the total amount of pH adjuster is from about 0.05 to about 15 wt. %, about 0.5 to about 10 wt. %, about 1 to about 5 wt. %, about 1.5 to about 4 wt. %, or about 2.0 to about 3 wt. %, including ranges and sub-ranges therebetween, based on the total weight of the composition.

### Methods of Use

The instant disclosure also relates to methods of using the cosmetic compositions described herein. For example, the cosmetic compositions can be used in a method that comprises applying the cosmetic compositions to the skin of humans. In some cases, the composition is applied to the face. Furthermore, the cosmetic composition can be used in methods for treating and/or repairing damage to skin (for example, damage from photoaging), and for diminishing the appearance of wrinkles, dark spots, and uneven skin texture of skin. The aforementioned methods are non-therapeutic.

The cosmetic composition may be applied once per day, twice per day, or more than once or twice per day. In some cases, the composition is applied in the evenings before bed. In other cases, the compositions are applies in the morning. In still other cases, the composition may be applied immediately after washing the skin. The compositions may be used once, or for a series of days, weeks, or months. For example, the compositions may be used daily for a period of 1, 2, 3, 4, 5, 6, 7, 8 or more weeks, or months.

### EXAMPLES

The following examples are provided primary for the purpose of elucidating the benefits achieved by embodiments of the disclosure. The examples serve to illustrate the technology without necessarily being limiting in nature.

### Example 1

A composition in accordance with the instant disclosure could be prepared as shown below:

TABLE 1
Inventive Example

| | | | Inven- |
|---|---|---|---|
| | | | tive Ex- |
| | | | ample 1 |
| | Cosmetic type | INCI US | |
| a | SKIN ENHANCER | HYDROXYETHYL UREA | 4.4 |
| | PENETRATOR | | |
| b | NATURAL | GLYCINE | 1 |
| | MOISTURING | SERINE | 0.1 |
| | FACTOR | ALANINE | 0.1 |
| c | N-SUBSTITUTED | HYDROXYETHYLPIPERAZINE | 0.2 |
| | AMINOSULFONIC | ETHANE SULFONIC ACID | |
| | ACID COMPOUND | | |
| | FATTY | CETEARYL ALCOHOL | 5 |
| | COMPOUND | | |
| | THICKENING | XANTHAN GUM | 2 |
| | AGENT | | |
| | SILICON | DIMETHICONE | 0.5 |
| | EMOLLIENT | ISOPROPYL PALMITATE | 4 |
| | EMULSIFIERS | GLYCERYL STEARATE (and) | 2.5 |
| | | PEG-100 STEARATE | |
| | PRESERVATIVES | | 4 |
| | SOLVENT | WATER | Q.S. |

### Example 2

Inventive Examples and Comparative Examples were tested to measure the enhanced penetration of the active ingredients through the skin. The enhanced penetration of the inventive and comparative Examples was assessed by following the procedure described below.

### Experimental Protocol for ATR-FTIR Measurements

1. Flash-frozen human skin from the same donor, purchased from a licensed supplier was used for all Raman experiments.

2. A 2 cm×2 cm piece of skin was thawed and cleaned.

3. 40 mg of formulation was applied to the skin surface and massaged with a glass rod for 60 seconds.

4. The skin was placed at the interface of a Franz diffusion cell with the acceptor chamber filled with distilled water.

5. The opening of the donor cell is covered with parafilm, and the cell is incubated at 32° C. for ˜14 hours (overnight).

6. After 14 hours, excess of formulation on the skin surface was gently blotted with a Kimwipe.

7. Skin was imaged by ATR-FTIR imaging spectroscopy to evaluate initial Natural Moisturizing Factor (NMF) deposition on the skin surface. In order to investigate the NMF penetration inside the stratum corneum (SC), 10 tape strips were applied and removed from the skin surface sequentially. 7 different depths inside the SC were obtained through this tape stripping method. This method allows the imaging of the whole SC profile. It allows also to specifically detect the signature band for tracking the penetration of hydroxyethyl urea as our humectant of interest for this disclosure.

TABLE 2
Inventive and Comparative Examples

| | | | A | B | C |
|---|---|---|---|---|---|
| | | | Comparative | Comparative | Inventive |
| | | | Ex. 1 | Ex. 2 | Ex. 2 |
| | | INCI US | | | |
| a | Skin Enhancer | Hydroxyethyl Urea | 4.4 | 4.4 | 4.4 |
| | Penetrator | | | | |
| b | Natural | Glycine | | 1 | 1 |
| | Moisturing | Alanine | | 0.1 | 0.1 |
| | Factor | Serine | | 0.1 | 0.1 |
| | | Sodium PCA | | 0.25 | 0.25 |
| c | N-Substituted | Hydroxyethylpiperazine | | | 0.2 |
| | Aminosulfonic | Ethane Sulfonic Acid | | | |
| | Acid Compound | (Hepes) | | | |
| | | Water | 95.6 | 94.1 | 93.9 |

### Results—Control

To visualize the skin distribution, specific ATR-FTIR images were generated. The goal of the control experiment was to visualize the skin distribution. In this experiment, 40 mg of each formula were applied to separate human skin samples. The samples were placed in a Franz diffusion cell for ˜14 hours. After 14 hours, any remaining formula was blotted from the surface of the skin samples. ATR-FTIR images were used to image the skin samples and evaluate the Amide I band area. The Amide band I is specific of the protein content. The higher was the value, the redder was the image and the higher was the protein content. See FIG. 3. As expected, the protein content did not present important variation in all the human skin samples. Only the ATR-FTIR images recorded 14 hours after topical application of the products on the skin surface (layer 0) presented a lower protein content as a significant amount of product was still present at the skin surface after the 14-hour treatment time.

### Results—Natural Moisturizing Factors Distribution

To visualize the Natural Moisturizer Factor (NMF) inside the stratum corneum, specific ATR-FTIR images were generated, and an evaluation of the 1060 cm⁻¹ band area was done. The 1060 cm⁻¹ band is specific of hydroxyethylurea. The higher was the value, the redder was the image and the higher was the hydroxyethyl urea content inside the human stratum corneum. The ATR-FTIR data demonstrated the distribution of the hydroxyethyl urea based on the 1060 cm⁻¹ band area into untreated human skin (control), and skin samples treated with the Inventive and Comparative Examples (C, A and B). See FIG. 4.

In this experiment, 40 mg of each formula were applied to separate human skin samples. The samples were placed in a Franz diffusion cell for ˜14 hours. After 14 hours, any remaining formula was blotted from the surface of the skin samples. ATR-FTIR was used to image the skin samples and evaluate the penetration of hydroxyethyl urea. To analyze the penetration of hydroxyethyl urea into the stratum corneum, tape strips were applied and removed from the skin 10 times, resulting in 7 different depths into the stratum corneum. When reading the results, the redder the image, the higher the hydroxyethyl urea content. As expected, no hydroxyethyl urea was detected in the untreated human skin sample (control). After 14-hour treatment time in the Fraz cell, hydroxyethyl urea was strongly detected at the surface of the human skin samples treated with formulation A, B and C. Based on the marker, the hydroxyethyl urea penetration was observed with the formulation C followed by the formulation A and B. It was demonstrated that formulation C was more efficient in terms of hydroxyethyl urea penetration. The hydroxyethyl urea was detected up to layer 8 out of 10. That results indicated that both hydroxyethyl urea and HEPEs were needed to have hydroxyethyl urea penetration.

These experiments surprisingly showed the penetration enhancement of hydroxyethyl urea in Formula C, containing hydroxyethyl urea, amino acids and hydroxyethylpiperazine ethane sulfonic acid, as the most efficient in hydroxyethyl urea penetration. Even a low level of hydroxyethylpiperazine ethane sulfonic acid helped the penetration of hydroxyethyl urea to penetrate the skin in the presence of amino acids (NMFs).

### Example 3

### Stability Data

The formulas were subjected to elevated temperatures and compared to a control jar to measure pH at set time points. The trials were conducted at 45° C. in large glass jars to accelerate color evolution and pH shifts. The results obtained are shown in Table 3 below.

TABLE 3

| | | 1 Week | | | | 2 Week | | | | 4 Week (1-month) | | | |
|---|---|---|---|---|---|---|---|---|---|---|---|---|---|
| | T₀ | 5° C. | Diff. | 45° C. | Diff. | 5° C. | Diff. | 45° C. | Diff. | 5° C. | Diff. | 45° C. | Diff. |
| A | 6.92 | 6.93 | 0.0 | 7.87 | 1.0 | 6.84 | −0.1 | 8.15 | 1.2 | 6.81 | −0.12 | 8.35 | 1.43 |
| B | 6.43 | 6.57 | 0.1 | 6.99 | 0.6 | 6.51 | 0.1 | 7.26 | 0.8 | 6.58 | 0.01 | 7.44 | 1.01 |
| C | 5.91 | 5.9 | 0.0 | 6.28 | 0.4 | 5.98 | 0.1 | 6.54 | 0.6 | 5.91 | 0.01 | 6.76 | 0.85 |

The stability protocol utilizes a 45° C. temperature check at 1-month and 2-month time points. The 1-month timepoint was used for this experiment. It was observed that Inventive Example Ex. 2 (C) had a more consistent pH compared to the Comparative Examples 1 and 2 (respectively A and B) that did not contain hydroxyethyl urea+NMFs+HEPES.

The pH is a key specification in cosmetic products. If pH shifts was too high/low, it could impact physical stability by having the ingredients crash out if the pH shifts outside of the range. Additionally, polymeric thickeners are dependent on pH so there can be viscosity changes if the pH shifts. pH is important for micro system too as a more neutral pH can lead to microbial growth. Although pH is important to this inventive example, pH is also important to chemical stability as certain actives are more stable at specific pH ranges.

A, B and C contained higher levels of hydroxyethyl urea which is known to have a pH shift at elevated temperatures. With a high amount of Natural Moisturizing Factors+HEPEs, the pH remained much more stable compared to A and B which did not contain the blend of hydroxyethyl urea+Natural Moisturizing Factors+HEPEs.

According to the results presented above, it was demonstrated that the compositions in accordance with the instant disclosure were surprisingly stable and unexpectedly enhanced the penetration of the hydroxyethyl urea and the Natural Moisturizer Factors even though the amount of hydroxyethyl urea was high.

While the disclosure has been described with reference to described embodiments, it will be understood by those skilled in the art that various changes may be made, and equivalents may be substituted for elements thereof without departing from the scope of the disclosure. In addition, many modifications may be made to adapt a particular situation or material to the teachings of the disclosure without departing from the essential scope thereof. Therefore, it is intended that the disclosure is not limited to the particular embodiment disclosed as the best mode contemplated for carrying out this disclosure, but that the disclosure will include all embodiments falling within the scope of the appended claims.

The term “INCI” is an abbreviation of International Nomenclature of Cosmetic Ingredients, which is a system of names provided by the International Nomenclature Committee of the Personal Care Products Council to describe personal care ingredients.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

All components and elements positively set forth in this disclosure can be negatively excluded from the claims. In other words, the cosmetic compositions of the instant disclosure can be free or essentially free of all components and elements positively recited throughout the instant disclosure.

In some instances, the cosmetic compositions of the present disclosure may be substantially free of non-incidental amounts of the ingredient(s) or compound(s) described herein. A non-incidental amount of an ingredient or compound is the amount of that ingredient or compound that is added into the cosmetic composition by itself. For example, a cosmetic composition may be substantially free of a non-incidental amount of an ingredient or compound, although such ingredient(s) or compound(s) may be present as part of a raw material that is included as a blend of two or more compounds.

Some of the various categories of components identified may overlap. In such cases where overlap may exist and the composition includes both components (or the composition includes more than two components that overlap), an overlapping compound does not represent more than one component. For example, tocopherol may be characterized as both a skin active agent and a preservative. If a particular composition includes both a skin active agent and a preservative, steareth-20 will serve only as the skin active agent or only as the preservative (tocopherol does not serve as both the skin active agent and preservative).

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

The expression “one or more” means “at least one” and thus includes individual components as well as mixtures/combinations.

Other than in the operating examples, or where otherwise indicated, all numbers expressing quantities of ingredients and/or reaction conditions may be modified in all instances by the term “about,” meaning within +/−5% of the indicated number.

The term “treat” (and its grammatical variations) as used herein refers to the application of the compositions of the present disclosure onto the surface of keratinous substrates such as hair on a user's head and/or body.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material. All of the components set forth herein may be optionally included or excluded from the compositions/method/kits. When excluded, the compositions/methods/kits may be free or essentially free of the component. For example, a particular composition may be free or essentially free of silicones.

1. A cosmetic composition comprising:
(a) hydroxyethylurea;
(b) at least three natural moisturizing factors;
(c) at least one N-substituted aminosulfonic acid compound.

2. The cosmetic composition of claim 1, wherein the hydroxyethylurea is present in an amount from about 1.5 to about 24 wt. %, based on a total weight of the cosmetic composition.

3. The cosmetic composition of claim 2, wherein the hydroxylethyl urea is present in an amount from about 1.8 to about 22 wt. %, based on the total weight of the cosmetic composition.

4. The cosmetic composition of claim 1 or 2, wherein the at least three natural moisturizing factors are selected from glycine, serine, alanine, sodium pca, or a combination thereof.

5. The cosmetic composition of any one of the above claims, wherein the at least three natural moisturizing factors are present in an amount from about 0.2 to about 9.5 wt. %, based on a total weight of the cosmetic composition.

6. The cosmetic composition of any one of the above claims, wherein the at least one N-substituted aminosulfonic acid compound is selected from N,N-bis[2-hydroxyethyl]-2-aminoethanesulfonic acid, N-2-hydroxyethylpiperazine-N′-2-ethanesulfonic acid, 3-[N-morpholino]propanesulfonic acid, piperazine-N,N′-bis[2-ethanesulfonic]acid, 3-[N-tris(hydroxymethyl)methylamino]-2-hydroxypropanesulfonic acid, 2-[N-morpholino]ethanesulfonic acid, N-(2-acetamido)-2-aminoethanesulfonic acid, and N-tris(hydroxymethyl)methyl-2-aminoethanesulfonic acid, or a combination thereof.

7. The cosmetic composition of any one of the above claims, wherein the at least one N-substituted aminosulfonic acid compound is present in an amount from about 0.15 to about 4.8 wt. %, based on the total weight of the cosmetic composition.

8. The cosmetic composition of any one of the above claims, wherein the pH of the composition is from about 4.5 to about 7.

9. The cosmetic composition of any one of the above claims, further comprising one or more compounds.

10. The cosmetic composition of any one of the above claims, further comprising one or more polymers.

11. The cosmetic composition of any one of the above claims, further comprising one or more surfactants.

12. The cosmetic composition of any one of the above claims, further comprising one or more water soluble solvents.

13. The cosmetic composition of any one of the above claims, further comprising at least one skin active agent.

14. The cosmetic composition of any one of the above claims, further comprising at least one skin active agent selected from antioxidants, free-radical scavengers, moisturizers, bleaching agents, liporegulators, anti-acne agents, antiseborrhoeic agents, anti-ageing agents, softeners, anti-wrinkle agents, anti-inflammatories, refreshing agents, cicatrizing agents, vascular protective agents, antibacterials, antifungals, exfoliating agents, antiperspirants, deodorants, skin conditioners, desensitizing agents, immunemodulators and nourishing agents, moisture absorbers, soothing active ingredients, sebum absorbing ingredients, or a combination thereof.

15. A cosmetic composition comprising:
(a) about 1.3 to about 25 wt. % of hydroxyethyl urea;
(b) about 0.1 to about 10 wt. % of one or more natural moisturizing factors; and
(c) about 0.1 to about 5 wt. % of hydroxyethylpiperazine ethane sulfonic acid;
wherein the composition is stable and the penetration of hydroxyethyl urea is improved, and all weight percentages are based on the total weight of the cosmetic composition.

16. A method for treating skin comprising applying the cosmetic composition of any one of the above claims to the skin.
